SEQUENCE LISTING (2 sequences)

SEQ ID NO: 1 - protein, 325 aa, Thermoanaerobacter tengcongensis
MGKVAVIFTGGTIAMKQDPNLKGAVPSLTGKDLLEIIPHRENVLEIETIEFGNYPSPHLTVELLEELREVVEKTLKRDEIKGVVITHGTDTLEETAYFLELTTNFDKPIVLTGAMKNSSEAGYDGPSNLIDSLTTASSEKSKKRGVLVVLNGEIHSAKDVTKTHTSSLDTFKSPETGPLGYVDSGNVYFYRNVEKRQYIPTKKLERRVSLLKVVFGMDDKLIRFLVENGERGIVIEGTGRGNVPPKVAEGIKYAISKGTVVVLVSRCPMGRVHPSYAYEGGGKHLESLGVIFGGNLSGQKARVKLMAALAYTSDYRKIREIFEEE

SEQ ID NO: 2 - DNA, 997 bp, Artificial sequence
  CpO sequence of putative Thermoanaerobacter tengcongensis asparaginase for expression in E.coli
catatgggcaaagtagcggtcatttttactggtggtacgattgcgatgaaacaagacccgaatctgaaaggtgcggttccgagcttgaccggcaaagatctgctggagattatcccacatcgcgaaaatgttctggaaatcgaaaccattgagttcggtaactatccgagcccgcacctgacggttgagctgctggaagaattgcgcgaagttgtcgagaaaaccctgaaacgtgacgaaatcaagggcgtggtgatcacccatggtacggacaccctggaagaaaccgcgtactttttagaactgaccaccaatttcgataagccgatcgtgctgacgggtgctatgaagaacagctccgaggccggctacgatggtccgtcaaatctgattgacagcctgacgacggcatcgagcgagaaaagcaaaaagcgtggcgtgctggttgtgttgaatggtgagatccatagcgctaaagacgtcaccaagacccacacctcttccctggacacctttaagagcccggaaaccggccctctgggctatgttgatagcggtaacgtctacttctaccgtaatgtcgagaagcgtcagtatattccgactaagaaactggagcgtcgcgtcagcctgttgaaagttgtttttggtatggatgacaagttgattcgtttcctggtggagaacggtgagcgtggtattgtgatcgaaggcacgggccgtggtaacgttccgccgaaagtcgccgagggtatcaaatatgcaatttccaagggtaccgttgtggtgctggtcagccgctgcccgatgggtcgtgtccacccgagctatgcgtacgagggtggcggcaagcacctggagtctctgggtgtgatctttggcggcaacctgagcggtcagaaagcgcgtgttaagctgatggcagccctggcatacacgagcgattaccgtaagatccgcgagattttcgaagaagagtaacctgcaggggcgcgcc

(19) United States
(12) Patent Application Publication
(10) Pub. No.: US 2018/0273929 A1
(43) Pub. Date: Sep. 27, 2018

(54) ASPARAGINASE

(71) Applicant: DSM IP Assets B.V., Heerlen (NL)

(72) Inventors: Jan Metske VAN DER LAAN, Echt (NL); Siew-loon OOI, Echt (NL); Aloysius Wilhelmus Rudolphus Hubertus TEUNISSEN, Echt (NL); Maarten Hotse WILBRINK, Echt (NL)

(73) Assignee: DSM IP ASSETS B.V., TE Heerlen (NL)

(21) Appl. No.: 15/762,327

(22) PCT Filed: Sep. 16, 2016
(86) PCT No.: PCT/EP2016/071971

§ 371 Date: Mar. 22, 2018

(30) Foreign Application Priority Data
Sep. 25, 2015 (EP) 15186978.1

(51) Int. Cl. C12N 9/82; A21D 2/26; A23L 5/20; A23L 19/15; A23L 7/104; A23L 7/135

(52) U.S. Cl. CPC C12N 9/82; C12Y 305/01001; A21D 2/267; A23L 5/25; A61K 38/00; A23L 7/104; A23L 7/135; A23V 2002/00; A23L 19/15

(57) ABSTRACT

The present invention relates to a polypeptide having asparaginase activity selected from the group consisting of: (i) a polypeptide having an amino acid sequence comprising the mature polypeptide sequence of SEQ ID NO: 1; (ii) a polypeptide comprising an amino acid sequence that has at least 50% sequence identity with the mature polypeptide sequence of SEQ ID NO: 1; (iii) a polypeptide encoded by a nucleic acid comprising a sequence that hybridizes under medium stringency conditions to the complementary strand of the mature polypeptide encoding sequence of SEQ ID NO: 2; and (iv) a polypeptide comprising an amino acid sequence encoded by a nucleic acid that has at least 50% sequence identity to the mature polypeptide coding sequence of SEQ ID NO: 2. The polypeptide may be used in the preparation of a food product.

### FIELD OF THE INVENTION

The present invention relates to a polypeptide having asparaginase activity and to a composition comprising such a polypeptide. The invention also relates to a nucleic acid encoding an asparaginase, to an expression vector comprising the nucleic acid and to a recombinant host cell comprising the nucleic acid or expression vector. The invention further relates to a method for the preparation of the polypeptide and to use of the polypeptide or composition in the production of a food product or to reduce the amount of acrylamide formed in a thermally processed food product based on an asparagine-containing raw material. The invention in addition relates to a process for the production of a food product involving at least one heating step and to a food product obtainable by such a process. Further, the invention relates to a dough comprising the polypeptide, to a method for the preparation of a dough and to a method for the preparation of a baked product. The invention also relates to the polypeptide or a composition for use in a method of treatment of the human or animal body by therapy.

### BACKGROUND TO THE INVENTION

The occurrence of acrylamide in a number of heated food products has been recognized for some time (Tareke et al. Chem. Res. Toxicol. 13, 517-522 (2000)). Since acrylamide is considered as probably carcinogenic for animals and humans, this finding resulted in world-wide concern. Further research revealed that considerable amounts of acrylamide are detectable in a variety of baked, fried and oven prepared common foods and it was demonstrated that the occurrence of acrylamide in food was the result of the heating process.

A pathway for the formation of acrylamide from amino acids and reducing sugars as a result of the Maillard reaction has been proposed by Mottram et al. Nature 419:448 (2002). According to this hypothesis, acrylamide may be formed during the Maillard reaction. During baking and roasting, the Maillard reaction is mainly responsible for the color, smell and taste. A reaction associated with the Maillard is the Strecker degradation of amino acids and a pathway to acrylamide was proposed. The formation of acrylamide became detectable when the temperature exceeded 120° C., and the highest formation rate was observed at around 170° C. When asparagine and glucose were present, the highest levels of acrylamide could be observed, while glutamine and aspartic acid only resulted in trace quantities.

The official migration limit in the EU for acrylamide migrating into food from food contact plastics is set at 10 ppb (10 micrograms per kilogram). Although no official limit is yet set for acrylamide that forms during cooking, the fact that a lot of products exceed this value, especially cereals, bread products and potato or corn based products, causes concern.

Several plant raw materials are known to contain substantial levels of asparagine. In potatoes asparagine is the dominant free amino acid (940 mg/kg, corresponding with 40% of the total amino-acid content) and in wheat flour asparagine is present as a level of about 167 mg/kg, corresponding with 14% of the total free amino acids pool (Belitz and Grosch in Food Chemistry—Springer New York, 1999). The fact that acrylamide is formed mainly from asparagine (combined with reducing sugars) may explain the high levels acrylamide in fried, oven-cooked or roasted plant products. Therefore, in the interest of public health, there is an urgent need for food products that have substantially lower levels of acrylamide or, preferably, are devoid of it.

A variety of solutions to decrease the acrylamide content has been proposed, either by altering processing variables, e.g. temperature or duration of the heating step, or by chemically or enzymatically preventing the formation of acrylamide or by removing formed acrylamide.

In several patent applications the use of asparaginase for decreasing the level of asparagine and thereby the amount of acrylamide formed has been disclosed. Suitable asparaginases for this purpose have been yielded from several fungal sources, as for example Aspergillus niger in WO2004/030468 and Aspergillus oryzae in WO04/032648.

Although all L-asparaginases catalyze the same chemical conversion, this does not mean that they are suitable for the same applications. Various applications will place different demands on the conditions under which the enzymes have to operate. Physical and chemical parameters that may influence the rate of an enzymatic conversion are the temperature (which has a positive effect on the chemical reaction rates, but may have a negative effect on enzyme stability), the moisture content, the pH, the salt concentration, the structural integrity of the food matrix, the presence of activators or inhibitors of the enzyme, the concentration of the substrate and products, etc.

Therefore there exists an ongoing need for improved asparaginases for several applications having improved properties, for example in higher temperature applications where thermostability may be advantagenous.

### SUMMARY OF THE INVENTION

The present invention is based on the identification of a polypeptide having asparaginase activity. The polypeptide may be derived from, for example, a microorganism of the genus Thermoanaerobacter such as from the species Thermoanaerobacter tengcongensis.

A polypeptide of the invention is preferably thermophilic, for example thermostable (i.e. capable of withstanding a thermal treatment in respect of its enzymatic activity) and/or thermoactive (i.e. only develops its full enzymatic activity at elevated temperature). A polypeptide of the invention may alternatively or additionally be one which is active across a broad pH range and/or at a relatively high or low pH.

Providing an asparaginase with improved thermophilic properties is an important way of broadening its application. Thermoactive and thermostable asparaginases have substantial advantages over other asparaginases. For instance, the conversion of the asparagine into aspartate can be conducted at comparatively high temperatures using thermoactive or thermostable asparaginases, and this results in a compatibility with processes in which high temperatures, in particular holding processes at high temperatures, still play a role. Moreover, the breakdown of asparagine at higher temperatures can be conducted at a higher reaction rate.

Asparaginases active at a broad pH range are also advantageous since it may be possible to use a polypeptide of the invention in different processes with widely differing pH ranges. It is also possible to use such a polypeptide in processes in which the pH value is subject to significant fluctuations in the process. Processes are also possible in which pH values from 5 to 10 occur.

The polypeptide of the invention having asparaginase activity may be used, in particular, in the preparation of a foodstuff, preferably to reduce the content of asparagine in the foodstuff. The reduction of the asparagine content preferably also causes the acrylamide content in the foodstuff to be reduced when the foodstuff is subjected to a subsequent thermal treatment.

Accordingly, the invention provides a polypeptide having asparaginase activity selected from the group consisting of:
i. a polypeptide having an amino acid sequence comprising the mature polypeptide sequence of SEQ ID NO: 1;
ii. a polypeptide comprising an amino acid sequence that has at least 50% sequence identity with the mature polypeptide sequence of SEQ ID NO: 1;
iii. a polypeptide encoded by a nucleic acid comprising a sequence that hybridizes under medium stringency conditions to the complementary strand of the mature polypeptide encoding sequence of SEQ ID NO: 2; and
iv. a polypeptide comprising an amino acid sequence encoded by a nucleic acid that has at least 50% sequence identity to the mature polypeptide coding sequence of SEQ ID NO: 2.

The invention also provides:
a composition comprising a polypeptide of the invention;
a nucleic acid encoding an asparaginase which comprises a sequence that has at least 50% sequence identity to the mature polypeptide encoding sequence of SEQ ID NO: 2;
a nucleic acid that is an isolated, substantially pure, pure, recombinant, synthetic or variant nucleic acid of a nucleic acid of the invention;
an expression vector comprising a nucleic acid of the invention operably linked to one or more control sequences that direct expression of the polypeptide in a host cell;
a recombinant host cell comprising a nucleic acid or an expression vector of the invention;
a method for the preparation of a polypeptide of the invention, which method comprises:
   cultivating a host cell according to claim 9 in a suitable fermentation medium under conditions that allow for production of the polypeptide; and, optionally,
   recovering the polypeptide;
use of a polypeptide of the invention, a polypeptide obtainable by a process of the invention or a composition of the invention in the production of a food product;
use of a polypeptide of the invention, a polypeptide obtainable by a process of the invention or a composition of the invention to reduce the amount of acrylamide formed in a thermally processed food product based on an asparagine-containing raw material;
a process for the production of a food product involving at least one heating step, which process comprises adding a polypeptide of the invention, a polypeptide obtainable by a process of the invention or a composition of the invention to an intermediate form of said food product in said production process, wherein the enzyme is added prior to or during said heating step in an amount that is effective in reducing the level of asparagine that is present in said intermediate form of said food product;
a food product obtainable by the process of the invention or by the use of the invention;
a dough comprising a polypeptide of the invention, a polypeptide obtainable by a process of the invention or a composition of the invention;
a method for the preparation of a dough, which method comprises combining: a polypeptide of the invention, a polypeptide obtainable by a process of the invention or a composition of the invention; and at least one dough ingredient.
a method for the preparation of a baked product, which method comprises the step of baking or frying a dough of the invention or a dough obtainable by a process of the invention for the preparation of a dough; and
a polypeptide of the invention, a polypeptide obtainable by a process of the invention or a composition of the invention for use in a method of treatment of the human or animal body by therapy.

### BRIEF DESCRIPTION OF THE DRAWINGS

FIG. 1 sets out the physical map of the asparaginase expression vector pAe7 containing the arabinose inducible promoter P_{BAD} and regulator araC, the kanamycin resistance gene Km(R) and the origin from pBR322. The NdeI and AscI sites are used to introduce the asparaginase gene.

FIG. 2 sets out the relative activity of the asparaginase polypeptide from Thermoanaerobacter tengcongensis at different temperatures.

FIG. 3 sets out the relative activity of the asparaginase polypeptide from Thermoanaerobacter tengcongensis at different pHs.

### DESCRIPTION OF THE SEQUENCE LISTING

SEQ ID NO: 1 sets out the amino acid sequence of an asparaginase polypeptide from Thermoanaerobacter tengcongensis (WP_011025341.1).

SEQ ID NO: 2 sets out the nucleotide sequence encoding the amino acid sequence of an asparaginase polypeptide from Thermoanaerobacter tengcongensis, codon-pair optimized for expression in E. coli. Start codon is at positions 4 to 6 and stop codon is at positions 979 to 981.

### DETAILED DESCRIPTION OF THE INVENTION

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

The term “complementary strand” can be used interchangeably with the term “complement”. The complementary strand of a nucleic acid can be the complement of a coding strand or the complement of a non-coding strand. When referring to double-stranded nucleic acids, the complement of a nucleic acid encoding a polypeptide refers to the complementary strand of the strand encoding the amino acid sequence or to any nucleic acid molecule containing the same. Typically, the reverse complementary strand is intended.

The term “control sequence” can be used interchangeably with the term “expression-regulating nucleic acid sequence”. The term as used herein refers to nucleic acid sequences necessary for and/or affecting the expression of an operably linked coding sequence in a particular host organism or in vitro. When two nucleic acid sequences are operably linked, they usually will be in the same orientation and also in the same reading frame. They usually will be essentially contiguous, although this may not be required. The expression-regulating nucleic acid sequences, such as inter alia appropriate transcription initiation, termination, promoter, leader, signal peptide, propeptide, prepropeptide, or enhancer sequences; Shine-Dalgarno sequence, repressor or activator sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion, can be any nucleic acid sequence showing activity in the host organism of choice and can be derived from genes encoding proteins, which are either endogenous or heterologous to a host cell. Each control sequence may be native or foreign to the nucleic acid sequence encoding the polypeptide. When desired, the control sequence may be provided with linkers for the purpose of introducing specific restriction sites facilitating ligation of the control sequences with the coding region of the nucleic acid sequence encoding a polypeptide. Control sequences may be optimized to their specific purpose.

The term “derived from” also includes the terms “originated from,” “obtained from,” “obtainable from,” “isolated from,” and “created from,” and generally indicates that one specified material find its origin in another specified material or has features that can be described with reference to the another specified material. As used herein, a substance (e.g., a nucleic acid molecule or polypeptide) “derived from” a microorganism preferably means that the substance is native to that microorganism.

As used herein, the term “endogenous” refers to a nucleic acid or amino acid sequence naturally occurring in a host cell.

The term “expression” includes any step involved in the production of the polypeptide including, but not limited to, transcription, post transcriptional modification, translation, post-translational modification, and secretion.

An “expression vector” comprises a polynucleotide coding for a polypeptide, operably linked to the appropriate control sequences (such as a promoter, and transcriptional and translational stop signals) for expression and/or translation in vitro, or in the host cell of the polynucleotide.

The expression vector may be any vector (e.g., a plasmid or virus), which can be conveniently subjected to recombinant DNA procedures and can bring about the expression of the polynucleotide. The choice of the vector will typically depend on the compatibility of the vector with the cell into which the vector is to be introduced. The vectors may be linear or closed circular plasmids. The vector may be an autonomously replicating vector, i.e. a vector, which exists as an extra-chromosomal entity, the replication of which is independent of chromosomal replication, e.g., a plasmid, an extra-chromosomal element, a mini-chromosome, or an artificial chromosome. Alternatively, the vector may be one which, when introduced into the host cell, is integrated into the genome and replicated together with the chromosome(s) into which it has been integrated. The integrative cloning vector may integrate at random or at a predetermined target locus in the chromosomes of the host cell. The vector system may be a single vector or plasmid or two or more vectors or plasmids, which together contain the total DNA to be introduced into the genome of the host cell, or a transposon.

A “host cell” as defined herein is an organism suitable for genetic manipulation and one which may be cultured at cell densities useful for industrial production of a target product, such as a polypeptide according to the present invention. A host cell may be a host cell found in nature or a host cell derived from a parent host cell after genetic manipulation or classical mutagenesis. Advantageously, a host cell is a recombinant host cell.

A host cell may be a prokaryotic, archaebacterial or eukaryotic host cell. A prokaryotic host cell may be, but is not limited to, a bacterial host cell. A eukaryotic host cell may be, but is not limited to, a yeast, a fungus, an amoeba, an algae, a plant, an animal cell, such as a mammalian or an insect cell.

The term “heterologous” as used herein refers to nucleic acid or amino acid sequences not naturally occurring in a host cell. In other words, the nucleic acid or amino acid sequence is not identical to that naturally found in the host cell.

The term “hybridization” means the pairing of substantially complementary strands of oligomeric compounds, such as nucleic acid compounds.

Hybridization may be performed under low, medium or high stringency conditions. Low stringency hybridization conditions comprise hybridizing in 6× sodium chloride/sodium citrate (SSC) at about 45° C., followed by two washes in 0.2×SSC, 0.1% SDS at least at 50° C. (the temperature of the washes can be increased to 55° C. for low stringency conditions). Medium stringency hybridization conditions comprise hybridizing in 6×SSC at about 45° C., followed by one or more washes in 0.2×SSC, 0.1% SDS at 60° C., and high stringency hybridization conditions comprise hybridizing in 6×SSC at about 45° C., followed by one or more washes in 0.2×SSC, 0.1% SDS at 65° C.

A nucleic acid or polynucleotide sequence is defined herein as a nucleotide polymer comprising at least 5 nucleotide or nucleic acid units. A nucleotide or nucleic acid refers to RNA and DNA. The terms “nucleic acid” and “polynucleotide sequence” are used interchangeably herein.

A “peptide” refers to a short chain of amino acid residues linked by a peptide (amide) bonds. The shortest peptide, a dipeptide, consists of 2 amino acids joined by single peptide bond.

The term “polypeptide” refers to a molecule comprising amino acid residues linked by peptide bonds and containing more than five amino acid residues. The term “protein” as used herein is synonymous with the term “polypeptide” and may also refer to two or more polypeptides. Thus, the terms “protein” and “polypeptide” can be used interchangeably. Polypeptides may optionally be modified (e.g., glycosylated, phosphorylated, acylated, farnesylated, prenylated, sulfonated, and the like) to add functionality. Polypeptides exhibiting activity in the presence of a specific substrate under certain conditions may be referred to as enzymes. It will be understood that, as a result of the degeneracy of the genetic code, a multitude of nucleotide sequences encoding a given polypeptide may be produced.

An “isolated nucleic acid fragment” is a nucleic acid fragment that is not naturally occurring as a fragment and would not be found in the natural state.

The term “isolated polypeptide” as used herein means a polypeptide that is removed from at least one component, e.g. other polypeptide material, with which it is naturally associated. The isolated polypeptide may be free of any other impurities. The isolated polypeptide may be at least 50% pure, e.g., at least 60% pure, at least 70% pure, at least 75% pure, at least 80% pure, at least 85% pure, at least 80% pure, at least 90% pure, or at least 95% pure, 96%, 97%, 98%, 99%, 99.5%, 99.9% as determined by SDS-PAGE or any other analytical method suitable for this purpose and known to the person skilled in the art. An isolated polypeptide may be produced by a recombinant host cell.

A “mature polypeptide” is defined herein as a polypeptide in its final form and is obtained after translation of a mRNA into polypeptide and post-translational modifications of said polypeptide. Post-translational modification include N-terminal processing, C-terminal truncation, glycosylation, phosphorylation and removal of leader sequences such as signal peptides, propeptides and/or prepropeptides by cleavage.

A “mature polypeptide coding sequence” means a polynucleotide that encodes a mature polypeptide (with reference to its amino acid sequence).

The term “nucleic acid construct” is herein referred to as a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct is synonymous with the term “expression cassette” when the nucleic acid construct contains all the control sequences required for expression of a coding sequence, wherein said control sequences are operably linked to said coding sequence.

The term “promoter” is defined herein as a DNA sequence that is bound by RNA polymerase and directs the polymerase to the correct downstream transcriptional start site of a nucleic acid sequence to initiate transcription. A promoter may also comprise binding sites for regulators.

The term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, underexpressed or not expressed at all. The term “recombinant” is synonymous with “genetically modified” and “transgenic”.

The terms “sequence identity” or “sequence homology” are used interchangeably herein. For the purpose of this invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleotides/bases or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.bioinformatics.nI/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.gov/.

The term “substantially pure” with regard to polypeptides refers to a polypeptide preparation which contains at the most 50% by weight of other polypeptide material. The polypeptides disclosed herein are preferably in a substantially pure form. In particular, it is preferred that the polypeptides disclosed herein are in “essentially pure form”, i.e. that the polypeptide preparation is essentially free of other polypeptide material. Optionally, the polypeptide may also be essentially free of non-polypeptide material such as nucleic acids, lipids, media components, and the like. Herein, the term “substantially pure polypeptide” is synonymous with the terms “isolated polypeptide” and “polypeptide in isolated form”. The term “substantially pure” with regard to polynucleotide refers to a polynucleotide preparation which contains at the most 50% by weight of other polynucleotide material. The polynucleotides disclosed herein are preferably in a substantially pure form. In particular, it is preferred that the polynucleotide disclosed herein are in “essentially pure form”, i.e. that the polynucleotide preparation is essentially free of other polynucleotide material. Optionally, the polynucleotide may also be essentially free of non-polynucleotide material such as polypeptides, lipids, media components, and the like. Herein, the term “substantially pure polynucleotide” is synonymous with the terms “isolated polynucleotide” and “polynucleotide in isolated form”.

A “synthetic molecule”, such as a synthetic nucleic acid or a synthetic polypeptide is produced by in vitro chemical or enzymatic synthesis. It includes, but is not limited to, variant nucleic acids made with optimal codon usage for host organisms of choice.

A synthetic nucleic acid may be optimized for codon use, preferably according to the methods described in WO2006/077258 and/or WO2008000632, which are herein incorporated by reference. WO2008/000632 addresses codon-pair optimization. Codon-pair optimization is a method wherein the nucleotide sequences encoding a polypeptide that have been modified with respect to their codon-usage, in particular the codon-pairs that are used, are optimized to obtain improved expression of the nucleotide sequence encoding the polypeptide and/or improved production of the encoded polypeptide. Codon pairs are defined as a set of two subsequent triplets (codons) in a coding sequence. Those skilled in the art will know that the codon usage needs to be adapted depending on the host species, possibly resulting in variants with significant homology deviation from SEQ ID NO: 1, but still encoding the polypeptide according to the invention.

As used herein, the terms “variant”, “derivative”, “mutant” or “homologue” can be used interchangeably. They can refer to either polypeptides or nucleic acids. Variants include substitutions, insertions, deletions, truncations, transversions, and/or inversions, at one or more locations relative to a reference sequence. Variants can be made for example by site-saturation mutagenesis, scanning mutagenesis, insertional mutagenesis, random mutagenesis, site-directed mutagenesis, and directed-evolution, as well as various other recombination approaches known to a skilled person in the art. Variant genes of nucleic acids may be synthesized artificially by known techniques in the art.

The invention relates to a polypeptide having asparaginase activity. Asparaginase (EC 3.5.1.1) is an enzyme that catalyzes the hydrolysis of aspargine to aspartic acid and ammonia. A polypeptide of the invention thus is capable of hydrolyzing asparagine to aspartic acid and ammonia.

A polypeptide of the invention is one having asparaginase activity and which is:
i. a polypeptide having an amino acid sequence comprising the mature polypeptide sequence of SEQ ID NO: 1;
ii. a polypeptide comprising an amino acid sequence that has at least 50% sequence identity with the mature polypeptide sequence of SEQ ID NO: 1;
iii. a polypeptide encoded by a nucleic acid comprising a sequence that hybridizes under medium stringency conditions to the complementary strand of the mature polypeptide encoding sequence of SEQ ID NO: 2 (or the corresponding wild-type sequence or a sequence codon optimized or codon pair optimized for expression in a heterologous organism, such as a Bacillus, for example Bacillus subtilis); or
iv. a polypeptide comprising an amino acid sequence encoded by a nucleic acid that has at least 50% sequence identity to the mature polypeptide coding sequence of SEQ ID NO: 2 (or the corresponding wild-type sequence or a sequence codon optimized or codon pair optimized for expression in a heterologous organism, such as a Bacillus, for example Bacillus subtilis).

The invention also provides a polypeptide of the invention which is:

i. a polypeptide comprising an amino acid sequence that has at least 60%, 70%, 75%, 80%, 85%, 90%, 91%, 92%, 93%, 94%, 95%, 96%, 97%, 98% or 99% sequence identity with the mature polypeptide sequence of SEQ ID NO: 1;

ii. a polypeptide encoded by a nucleic acid comprising a sequence that hybridizes under high stringency conditions to the complementary strand of the mature polypeptide encoding sequence of SEQ ID NO: 2 (or the corresponding wild-type sequence or a sequence codon optimized or codon pair optimized for expression in a heterologous organism, such as a Bacillus, for example Bacillus subtilis); or

ii. a polypeptide comprising an amino acid sequence encoded by a nucleic acid that has at least 60%, 70%, 75%, 80%, 85%, 90%, 91%, 92%, 93%, 94%, 95%, 96%, 97%, 98% or 99% sequence identity to the mature polypeptide coding sequence of SEQ ID NO: 2 (or the corresponding wild-type sequence or a sequence codon optimized or codon pair optimized for expression in a heterologous organism, such as a Bacillus, for example Bacillus subtilis).

The invention also relates to polypeptides which are isolated, substantially pure, pure, recombinant, synthetic or variant polypeptides of such polypeptides.

A polypeptide of the invention may be derivable from an organism of the genus Thermoanaerobacter, such as from Thermoanaerobacter tengcongensis. The wording “derived” or “derivable” from with respect to the origin of a polypeptide of the invention means that when carrying out a BLAST search with a polypeptide according to the present invention, the polypeptide according to the present invention may be derivable from a natural source, such as a microbial cell, of which an endogenous polypeptide shows the highest percentage homology or identity with the polypeptide as disclosed herein.

Preferably, a polypeptide of the invention may be a polypeptide that has least 50%, 60%, 70%, 75%, 80%, 85%, 86%, 87%, 88%, 89%, 90%, 91%, 92%, 93%, 94%, 95%, 96%, 97%, 98% or 99% sequence identity to the mature polypeptide sequence of SEQ ID NO: 1.

When produced in a heterologous host, a polypeptide of the invention may be produced in a form which omits the methionine at position 1 in which case a polypeptide of the invention may be a polypeptide that has least 50%, 60%, 70%, 75%, 80%, 85%, 86%, 87%, 88%, 89%, 90%, 91%, 92%, 93%, 94%, 95%, 96%, 97%, 98% or 99% sequence identity to the polypeptide sequence of amino acids 1 to 325 of SEQ ID NO: 1.

The mature polypeptide sequence typically has the amino acid sequence of amino acids 1 to 325 of SEQ ID NO: 1.

A polypeptide according to the present invention may be encoded by any suitable polynucleotide sequence. Typically a polynucleotide sequence is codon optimized, or a codon pair optimized sequence for expression of a polypeptide as disclosed herein in a particular host cell. A polypeptide of the invention may be encoded by a polynucleotide sequence that comprises appropriate control sequences and/or signal sequences, for example for secretion.

A polypeptide of the invention may be encoded by a polynucleotide that hybridizes under medium stringency, preferably under high stringency conditions to the complementary strand of the mature polypeptide coding sequence of SEQ ID NO: 2 (or the corresponding wild-type sequence or a sequence codon optimized or codon pair optimized for expression in a heterologous organism, such as a Bacillus, for example Bacillus subtilis).

A polypeptide of the invention may also be encoded by a nucleic acid that has at least 80%, 85%, 90%, 91%, 92%, 93%, 94%, 95%, 96%, 97%, 98%, 99% or 100% identity to a mature polypeptide coding sequence of SEQ ID NO: 2 (or the corresponding wild-type sequence or a sequence codon optimized or codon pair optimized for expression in a heterologous organism, such as a Bacillus, for example Bacillus subtilis).

A polypeptide of the invention may also be a variant of a mature polypeptide of SEQ ID NO: 1, comprising a substitution, deletion and/or insertion at one or more positions of the mature polypeptide SEQ ID NO: 1. A variant of the mature polypeptide of SEQ ID NO: 1 may be an amino acid sequence that differs in 1, 2, 3, 4, 5, 6, 7, 8, 9, 10, 11 or 12 amino acids from the amino acids of the mature polypeptide of SEQ ID NO: 1.

In one embodiment the present invention features a biologically active fragment of a polypeptide as disclosed herein.

Biologically active fragments of a polypeptide of the invention include polypeptides comprising amino acid sequences sufficiently identical to or derived from the amino acid sequence of the asparaginase protein (e.g., the mature amino acid sequence of SEQ ID NO: 1), which include fewer amino acids than the full length protein but which exhibits at least one biological activity of the corresponding full-length protein. Typically, biologically active fragments comprise a domain or motif with at least one activity of the asparaginase protein. A biologically active fragment may for instance comprise a catalytic domain. A biologically active fragment of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the biological activities of the native form of a polypeptide of the invention.

The invention also features nucleic acid fragments which encode the above biologically active fragments of the asparaginase protein.

A polypeptide according to the present invention may be a fusion protein. Techniques for producing fusion polypeptides are known in the art, and include ligating the coding sequences encoding the polypeptides so that they are in frame. Expression of the fused polypeptide is under control of the same promoter (s) and terminator. The hybrid polypeptides may comprise a combination of partial or complete polypeptide sequences obtained from at least two different polypeptides wherein one or more may be heterologous to a host cell. Such fusion polypeptides from at least two different polypeptides may comprise a binding domain from one polypeptide, operably linked to a catalytic domain from a second polypeptide. Examples of fusion polypeptides and signal sequence fusions are for example as described in WO2010/121933, WO2013/007820 and WO2013/007821.

A polypeptide of the invention may be a naturally occurring polypeptide or a genetically modified or recombinant polypeptide.

A polypeptide of the invention may be purified. Purification of proteins is known to a skilled person in the art.

A polypeptide of the invention may preferably be thermostable and/or thermoactive. Additionally or alternatively, a polypeptide of the invention may be active across a broad pH range and/or active at a relatively high or low pH.

A polypeptide of the invention may be thermostable. “Thermostable” herein means that a polypeptide of the invention may have a residual asparaginase activity of at least 50% after an incubation period of 5 min at 50° C. A polypeptide of the invention may have a residual asparaginase activity of at least 50% after an incubation period of 5 min at 55° C., 60° C., 65° C., 70° C. or at a higher temperature.

“Residual activity” herein means any specific/volumetric enzymatic activity that an enzyme has after a specific incubation duration at a specific temperature compared with the original specific/volumetric activity in the range of its temperature optimum under otherwise identical reaction conditions (pH, substrate etc.). The specific/volumetric activity of an enzyme means a specific amount of a converted substrate (for example in μmol) per unit time (for example in minutes) per enzyme amount (for example in mg or ml). The residual activity of an enzyme results from the specific/volumetric activity of the enzyme after the aforementioned incubation duration divided by the original specific/volumetric activity expressed as a percentage (%). In this case, the specific activity of an enzyme may be indicated in U/mg and the volumetric activity of an enzyme may be indicated in U/ml. Alternatively, the specific/volumetric activity of an enzyme can also be indicated in katal/mg or katal/ml in the sense of the description.

The term “enzymatic activity”, sometimes also referred to as “catalytic activity” or “catalytic efficiency”, is generally known to the person skilled in the art and refers to the conversion rate of an enzyme and is usually expressed by means of the ratio kₖₐₜ/K_{M}, wherein kₖₐₜ is the catalytic constant (also referred to as turnover number) and the K_{M} value corresponds to the substrate concentration, at which the reaction rate lies at half its maximum value. Alternatively, the enzymatic activity of an enzyme can also be specified by the specific activity (μmol of converted substrate×mg⁻¹×min⁻¹; cf. above) or the volumetric activity (μmol of converted substrate×ml⁻¹×min⁻¹; cf. above).

Reference can also be made to the general literature such as Structure and Mechanism in Protein Science: A guide to enzyme catalysis and protein folding, Alan Fersht, W. H. Freeman, 1999; Fundamentals of Enzyme Kinetics, Athel Cornish-Bowden, Wiley-Blackwell 2012 and Voet et al., “Biochemie” [Biochemistry], 1992, VCH-Verlag, Chapter 13, pages 331-332 with respect to enzymatic activity.

Thus, a thermostable polypeptide of the invention may have a residual activity of at least 50%, at least 60%, at least 70%, at least 80%, at least 90% or higher under the following conditions as set out in Table 1. In Table 1 for example condition 4B means that the asparaginase after 30 minutes at 60° C. has a residual activity of at least 75%, such as at least 80%, for example at least 90%.

TABLE 1
Conditions to determine residual activity.

| | |
|---|---|
| Time | Temperature (degrees Celsius) |

| Nr | (min) | A | B | C | D | E | F |
|---|---|---|---|---|---|---|---|
| 1 | 5 | 50 | 60 | 70 | 80 | 90 | 100 |
| 2 | 10 | 50 | 60 | 70 | 80 | 90 | 100 |
| 3 | 20 | 50 | 60 | 70 | 80 | 90 | 100 |
| 4 | 30 | 50 | 60 | 70 | 80 | 90 | 100 |
| 5 | 40 | 50 | 60 | 70 | 80 | 90 | 100 |
| 6 | 50 | 50 | 60 | 70 | 80 | 90 | 100 |
| 7 | 60 | 50 | 60 | 70 | 80 | 90 | 100 |
| 8 | 70 | 50 | 60 | 70 | 80 | 90 | 100 |

Preferably, a polypeptide of the invention has a residual activity in the range of from 75% to 100%, such as 75% to 90% under the conditions specified above.

A polypeptide of the invention having asparaginase activity is preferably thermoactive. “Thermoactive” herein means that the temperature optimum of such a polypeptide is at least about 50° C., at least about 55° C., at least about 60° C., at least about 65° C., at least about 70° C. or higher.

Thermoactivity may be determined as set out in Example 2.

The term “temperature optimum” is generally known to the skilled person and relates to the temperature range at which an enzyme exhibits its maximum enzymatic activity. Reference can be made in association with this to the relevant literature such as Enzyme Assays: A Practical Approach, Robert Eisenthal, Michael J. Danson, Oxford University Press 2002; Voet et al., “Biochemie”, 1992, VCH-Verlag, Chapter 13, page 331; I. H. Segel, Enzyme Kinetics: Behavior and Analysis of Rapid Equilibrium and Steady-State Enzyme Systems, Wiley Interscience, 1993; and A. G. Marangoni, Enzyme Kinetics: A Modern Approach, Wiley Interscience, 2002.

Herein, the temperature optimum is preferably understood to be the temperature range, in which a polypeptide of the invention has at least 80%, preferably at least 90% of the maximum enzymatic activity under otherwise constant reaction conditions.

The temperature optimum of a polypeptide according to the invention preferably lies in the range of from 60° to 130° C., such as in the range of from 70° to 120° C., for example in the range of 75° to 110° C. or in the range of from 80° to 100° C.

Thus, a polypeptide of the invention is preferably thermostable (i.e. capable of withstanding a thermal treatment in respect of its enzymatic activity) and/or thermoactive (i.e. only develops its full enzymatic activity at elevated temperature).

At a temperature of from 60° to 120° C., such as from 65° to 110° C., such as from 70° to 100° C., such as from 75° to 100° C. or from 80° to 90° C., a polypeptide of the invention may have a specific activity of preferably at least 100, more preferred at least 200, further preferred at least 300, further preferred at least 500, most preferred at least 800 and in particular at least 100 units/mg, wherein 1 unit is defined as the amount of the polypeptide that releases 1.0 μmol of ammonia per minute from L-asparagine under the conditions set out in the Examples

A polypeptide of the invention may be active at a relatively high pH. Accordingly, a polypeptide of the invention may have a pH optimum which is higher than the wild-type asparaginase from A. niger (as disclosed in WO2004/030468) which has a pH optimum of from pH 4 to pH 5. A polypeptide of the invention may be more alkaliphilic than such a wild-type enzyme, i.e. may, for example, have a pH optimum of from pH 5 to pH 11, such as from pH 6 to pH 10. Optionally a variant protein of the invention may be more acidophilic than the wild type asparaginase from A. niger.

The term “pH optimum” is generally known to the skilled person and relates to the pH range, in which an enzyme has its maximum enzymatic activity. Reference can be made in association with this to the relevant literature such as Enzyme Assays: A Practical Approach, Robert Eisenthal, Michael J. Danson, Oxford University Press 2002 and Voet et al., “Biochemie”, 1992, VCH-Verlag, Chapter 13, page 331. Herein, the term pH optimum is typically understood to mean the pH range, in which the amidohydrolase used according to the invention has at least 80%, preferably at least 90% of the maximum enzymatic activity under otherwise constant reaction conditions.

A polypeptide according to the invention may have a pH, which may be higher than the pH optimum and at which at least 50% of the asparaginase activity is still present, (hereafter indicated as alkaline pH), which is higher than that of the wild type asparaginase from A. niger. Thus, a polypeptide of the invention may have an alkaline pH at which at least 50% of the activity (at the pH optimum) is observed which may at least pH 7.0.

A polypeptide of the invention may be active over a very broad pH range. In the range from pH 5 to pH 10, a polypeptide of the invention may preferably have an activity of at least 10% of the maximum activity. As a result of this, it may possible to use a polypeptide of the invention in different processes with widely differing pH ranges. It is also possible to use it in processes in which the pH value is subject to significant fluctuations in the process. Processes are also possible in which pH values from 5 to 10 occur.

Over the entire pH range of from pH 5 to pH 10, a polypeptide of the invention has an activity of at least 10%, more preferred at least 15%, further preferred at least 20%, most preferred at least 25% and in particular at least 30% compared to the maximum activity, i.e. to the maximum activity with the optimum pH value under otherwise identical conditions, preferably at optimum temperature and concentration.

The invention further provides a nucleic acid encoding an asparaginase which comprises a sequence that has at least 50% sequence identity to the mature polypeptide encoding sequence of SEQ ID NO: 2.

A nucleic acid of the invention may comprise a polynucleotide sequence encoding a polypeptide of the invention which has at least 60%, 70%, 75%, 80%, 85%, 90%, 95%, 96%, 97%, 98%, or 99% sequence identity to SEQ ID NO: 2, or to the mature polypeptide coding sequence of either thereof.

A polynucleotide sequence of the invention may comprise SEQ ID NO: 2 or may comprise the mature polypeptide coding sequence of either thereof.

A nucleic acid of the invention may be an isolated, substantially pure, pure, recombinant, synthetic or variant nucleic acid of the nucleic acid of SEQ ID NO: 2. A variant nucleic acid sequence may for instance have at least 80% sequence identity to SEQ ID NO: 2.

The invention also provides a nucleic acid construct comprising a nucleic acid of the invention. An expression vector is also provide which comprises a nucleic acid of the invention or a nucleic acid of the invention operably linked to one or more control sequences that direct expression of the polypeptide in a host cell.

There are several ways of inserting a nucleic acid into a nucleic acid construct or an expression vector which are known to a skilled person in the art, see for instance Sambrook & Russell, Molecular Cloning: A Laboratory Manual, 3rd Ed., CSHL Press, Cold Spring Harbor, N.Y., 2001. It may be desirable to manipulate a nucleic acid encoding a polypeptide of the present invention with control sequences, such as promoter and terminator sequences.

A promoter may be any appropriate promoter sequence suitable for a eukaryotic or prokaryotic host cell, which shows transcriptional activity, including mutant, truncated, and hybrid promoters, and may be obtained from polynucleotides encoding extracellular or intracellular polypeptides either endogenous (native) or heterologous (foreign) to the cell. The promoter may be a constitutive or inducible promoter. An inducible promoter may be, for example, a starch inducible promoter.

In the invention, bacteria may preferably be used as host cells for the expression of a polypeptide of the invention, in particular Bacilli. Suitable inducible promoters useful in such host cells include promoters that may be regulated primarily by an ancillary factor such as a repressor or an activator. The repressors are sequence-specific DNA binding proteins that repress promoter activity. The transcription can be initiated from this promoter in the presence of an inducer that prevents binding of the repressor to the operator of the promoter. Examples of such promoters from Gram-positive microorganisms include, but are not limited to, gnt (gluconate operon promoter); penP from Bacillus licheniformis; glnA (glutamine synthetase); xylAB (xylose operon); araABD (L-arabinose operon) and P_{spac} promoter, a hybrid SPO1/lac promoter that can be controlled by inducers such as isopropyl-β-D-thiogalactopyranoside [IPTG] ((Yansura D. G., Henner D. J. Proc Natl Acad Sci USA. 1984 81(2):439-443). Activators are also sequence-specific DNA binding proteins that induce promoter activity. Examples of such promoters from Gram-positive microorganisms include, but are not limited to, two-component systems (PhoP-PhoR, DegU-DegS, SpoOA-Phosphorelay), LevR, Mry and GltC. Production of secondary sigma factors can be primarily responsible for the transcription from specific promoters. Examples from Gram-positive microorganisms include, but are not limited to, the promoters activated by sporulation specific sigma factors: σ^{F}, σ^{E}, σ^{G} and σ^{K} and general stress sigma factor, σ^{B}. The σ^{B}-mediated response is induced by energy limitation and environmental stresses (Hecker M, VOlker U. Mol Microbiol. 1998; 29(5):1129-1136.). Attenuation and antitermination also regulates transcription. Examples from Gram-positive microorganisms include, but are not limited to, trp operon and sacB gene. Other regulated promoters in expression vectors are based the sacR regulatory system conferring sucrose inducibility (Klier A F, Rapoport G. Annu Rev Microbiol. 1988; 42:65-95).

Strong constitutive promoters are well known and an appropriate one may be selected according to the specific sequence to be controlled in the host cell. Suitable inducible promoters useful in bacteria, such as Bacilli, include: promoters from Gram-positive microorganisms such as, but are not limited to, SP01-26, SP01-15, veg, pyc (pyruvate carboxylase promoter), and amyE. Examples of promoters from Gram-negative microorganisms include, but are not limited to, tac, tet, trp-tet, lpp, lac, lpp-lac, laclq, T7, T5, T3, gal, trc, ara, SP6, λ-P_{R}, and λ-P_{L}.

Additional examples of promoters useful in bacterial cells, such as Bacilli, include the α-amylase and SPo2 promoters as well as promoters from extracellular protease genes.

The promoter sequences may be obtained from a bacterial source. In a more preferred embodiment, the promoter sequences may be obtained from a gram positive bacterium such as a Bacillus strain, e.g., Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis; or a Streptomyces strain, e.g., Streptomyces lividans or Streptomyces murinus; or from a gram negative bacterium, e.g., E. coli or Pseudomonas sp.

An example of a suitable promoter for directing the transcription of a polynucleotide sequence of the present invention is the promoter obtained from the E. coli lac operon. Another example is the promoter of the Streptomyces coelicolor agarase gene (dagA). Another example is the promoter of the Bacillus lentus alkaline protease gene (aprH). Another example is the promoter of the Bacillus licheniformis alkaline protease gene (subtilisin Carlsberg gene). Another example is the promoter of the Bacillus subtilis levansucrase gene (sacB). Another example is the promoter of the Bacillus subtilis alphaamylase gene (amyF). Another example is the promoter of the Bacillus licheniformis alphaamylase gene (amyL). Another example is the promoter of the Geobacillus stearothermophilus glucan 1,4-□-maltohydrolase gene (amyM). Another example is the promoter of the Bacillus amyloliquefaciens alpha-amylase gene (amyQ). Another example is a “consensus” promoter having the sequence TTGACA for the “−35” region and TATAAT for the “−10” region. Another example is the promoter of the Bacillus licheniformis penicillinase gene (penP). Another example are the promoters of the Bacillus subtilis xylA and xylB genes.

Preferably the promoter sequence is from a highly expressed gene. Examples of preferred highly expressed genes from which promoters may be selected and/or which are comprised in preferred predetermined target loci for integration of expression constructs, include but are not limited to genes encoding glycolytic enzymes such as triose-phosphate isomerases (TPD, glyceraldehyde-phosphate dehydrogenases (GAPDH), phosphoglycerate kinases (PGK), pyruvate kinases (PYK or PKI), alcohol dehydrogenases (ADH), as well as genes encoding amylases, glucoamylases, proteases, xylanases, cellobiohydrolases, β-galactosidases, alcohol (methanol) oxidases, elongation factors and ribosomal proteins. Specific examples of suitable highly expressed genes include e. g. the LAC4 gene from Kluyveromyces sp., the methanol oxidase genes (AOX and MOX) from Hansenula and Pichia, respectively, the glucoamylase (glaA) genes from A. niger and A. awamori, the A. oryzae TAKA-amylase gene, the A. nidulans gpdA gene and the T. reesei cellobiohydrolase genes.

Promoters which can be used in yeasts include e.g. promoters from glycolytic genes, such as the phosphofructokinase (PFK), triose phosphate isomerase (TPI), glyceraldehyde-3-phosphate dehydrogenase (GPD, TDH3 or GAPDH), pyruvate kinase (PYK), phosphoglycerate kinase (PGK) promoters from yeasts or filamentous fungi; more details about such promoters from yeast may be found in (WO 93/03159). Other useful promoters are ribosomal protein encoding gene promoters, the lactase gene promoter (LAC4), alcohol dehydrogenase promoters (ADHI, ADH4, and the like), and the enolase promoter (ENO). Other promoters, both constitutive and inducible, and enhancers or upstream activating sequences will be known to those of skill in the art. The promoters used in the host cells of the invention may be modified, if desired, to affect their control characteristics. Suitable promoters in this context include both constitutive and inducible natural promoters as well as engineered promoters, which are well known to the person skilled in the art. Suitable promoters in eukaryotic host cells may be GAL7, GAL10, or GAL1, CYC1, HIS3, ADHI, PGL, PH05, GAPDH, ADC1, TRP1, URA3, LEU2, ENO1, TPI1, and AOX1. Other suitable promoters include PDC1, GPD1, PGK1, TEF1, and TDH3. Examples of carbohydrate inducible promoters which can be used are GAL promoters, such as GAL1 or GAL10 promoters.

Promoters suitable in filamentous fungi are promoters which may be selected from the group, which includes but is not limited to promoters obtained from the polynucleotides encoding A. oryzae TAKA amylase, Rhizomucor miehei aspartic proteinase, Aspergillus gpdA promoter, A. niger neutral alpha-amylase, A. niger acid stable alpha-amylase, A. niger or A. awamori glucoamylase (glaA), A. niger or A. awamori endoxylanase (xlnA) or beta-xylosidase (xlnD), T. reesei cellobiohydrolase I (CBHI), R. miehei lipase, A. oryzae alkaline protease, A. oryzae triose phosphate isomerase, A. nidulans acetamidase, Fusarium venenatum amyloglucosidase (WO 00/56900), Fusarium venenatum Dania (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the polynucleotides encoding A. niger neutral alpha-amylase and A. oryzae triose phosphate isomerase), and mutant, truncated, and hybrid promoters thereof.

All of the above-mentioned promoters are readily available in the art.

Any terminator which is functional in a cell as disclosed herein may be used, which are known to a skilled person in the art.

Examples of suitable terminator sequences in filamentous fungi include terminator sequences of a filamentous fungal gene, such as from Aspergillus genes, for instance from the gene A. oryzae TAKA amylase, the genes encoding A. niger glucoamylase (glaA), A. nidulans anthranilate synthase, A. niger alpha-glucosidase, trpC and/or Fusarium oxysporum trypsin-like protease.

The invention also relates to a vector which comprises a nucleic acid of the invention, said vector comprises at least an autonomous replication sequence and a nucleic acid as described herein.

The vector may be any vector (e.g. a plasmid or a virus), which can be conveniently subjected to recombinant DNA procedures. The choice of the vector will typically depend on the compatibility of the vector with the host cell into which the vector is to be introduced. Preferably, the vector is a plasmid. The vector may be a linear or a closed circular plasmid. The vector may further comprise a, preferably non-selective, marker that allows for easy determination of the vector in the host cell. Suitable markers include GFP and DsRed. The chance of gene conversion or integration of the vector into the host genome is preferably minimized. The vector according to the invention may be an extra-chromosomal vector. Such a vector preferably lacks significant regions of homology with the genome of the host to minimize the chance of integration into the host genome by homologous recombination. The person skilled in the art knows how to construct a vector with minimal chance of integration into the genome. This may be achieved by using control sequences, such as promoters and terminators, which originate from another species than the host species. Other ways of reducing homology are by modifying codon usage and introduction of silent mutations. The person skilled in the art knows that the type of host cell, the length of the regions of homology to the host cell genome present in the vector, and the percentage of homology between said regions of homology in the vector and the host chromosome will determine whether and in which amount the vector will integrate into the host cell genome.

The autonomous replication sequence may be any suitable sequence available to the person skilled in the art that allows for plasmid replication that is independent of chromosomal replication.

The origin of replication may be any plasmid replicator mediating autonomous replication that functions in a cell. The term “origin of replication” or “plasmid replicator” is defined herein as a nucleotide sequence that enables a plasmid or vector to replicate in vivo. Examples of bacterial origins of replication are the origins of replication of plasmids pBR322, pUC19, pACYC177, and pACYC184 permitting replication in E. coli, RSF1010 permitting replication in Pseudomonas is described, e.g., by F. Heffron et al., in Proc. Nat'l Acad. Sci. USA 72(9):3623-27 (September 1975), and pUB110, pE194, pTA1060, and pAMß1 permitting replication in Bacillus.

Preferably, the autonomous replication sequence used in filamentous fungi is the AMA1 replicon (Gems et al., 1991 Gene. 98(1):61-7). Telomeric repeats may also result in autonomous replication (In vivo linearization and autonomous replication of plasmids containing human telomeric DNA in Aspergillus nidulans, Aleksenko et al. Molecular and General Genetics MGG, 1998-Volume 260, Numbers 2-3, 159-164, DOI: 10.1007/s004380050881). CEN/ARS sequences and 3p vector sequences from yeast may also be suitable.

A vector or expression construct for a given host cell may thus comprise the following elements operably linked to each other in a consecutive order from the 5′-end to 3′-end relative to the coding strand of the sequence encoding the compound of interest or encoding a compound involved in the synthesis of the compound of interest: (1) a promoter sequence capable of directing transcription of a nucleic acid of the invention; (2) optionally a sequence to facilitate the translation of the transcribed RNA, for example a ribosome binding site (also indicated as Shine Delgarno sequence) in prokaryotes, or a Kozak sequence in eukaryotes (3) optionally, a signal sequence capable of directing secretion of the asparaginase encoded by the nucleic acid of the invention from the given host cell into a culture medium; (4) a nucleic acid of the invention, as described herein; and preferably also (5) a transcription termination region (terminator) capable of terminating transcription downstream of the nucleic acid of the invention. The vector may comprise these and/or other control sequences as described herein.

Downstream of a nucleic acid of the invention there may be a 3′-untranslated region containing one or more transcription termination sites (e. g. a terminator, herein also referred to as a stop codon). The origin of the terminator is not critical. The terminator can, for example, be native to the DNA sequence encoding the polypeptide. However, preferably a bacterial terminator is used in bacterial host cells and a filamentous fungal terminator is used in filamentous fungal host cells. More preferably, the terminator is endogenous to the host cell (in which the nucleotide sequence encoding the polypeptide is to be expressed). In the transcribed region, a ribosome binding site for translation may be present. The coding portion of the mature transcripts expressed by the constructs will include a start codon, usually AUG (or ATG), but there are also alternative start codons, such as for example GUG (or GTG) and UUG (or TTG), which are used in prokaryotes. Also a stop or translation termination codon is appropriately positioned at the end of the polypeptide to be translated.

Enhanced expression of an asparaginase of the invention may also be achieved by the selection of homologous and heterologous regulatory regions, e. g. promoter, secretion leader and/or terminator regions, which may serve to increase expression and, if desired, secretion levels of the protein of interest from the expression host and/or to provide for the inducible control of the expression of a compound of interest or a compound involved in the synthesis of a compound of interest.

The vector comprising at least an autonomous replication sequence and a nucleic acid of the invention, also referred to herein as “vector (or expression vector) of the invention” can be designed for expression of the nucleic acid in a prokaryotic or a eukaryotic cell. For example, an asparaginase of the invention can be produced in bacterial cells such as E. coli or Bacilli, insect cells (using baculovirus expression vectors), fungal cells, such as yeast cells, or mammalian cells. Suitable host cells are discussed herein and further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

In order to identify and select cells which harbour a nucleic acid and/or vector of the invention, a gene that encodes a selectable marker (e.g., resistance to antibiotics) is optionally introduced into the vector and/or host cells along with the nucleic acid of the invention. Preferred selectable markers include, but are not limited to those which confer resistance to drugs or which complement a defect in the host cell.

Such markers include ATP synthetase, subunit 9 (oliC), orotidine-5′-phosphatedecarboxylase (pvrA), the bacterial G418 resistance gene (this may also be used in yeast, but not in fungi), the ampicillin resistance gene (E. coli), resistance genes for neomycin, kanamycin, tetracycline, spectinomycin, erythromycin, chloramphenicol, phleomycin (Bacillus) and the E. coli uidA gene, coding for β-glucuronidase (GUS). Vectors may be used in vitro, for example for the production of RNA or used to transfect or transform a host cell.

They also include e. g. versatile marker genes that can be used for transformation of most filamentous fungi and yeasts such as acetamidase genes or cDNAs (the amdS, niaD, facA genes or cDNAs from A. nidulans, A. oryzae or A. niger), or genes providing resistance to antibiotics like G418, hygromycin, bleomycin, kanamycin, methotrexate, phleomycin orbenomyl resistance (benA). Alternatively, specific selection markers can be used such as auxotrophic markers which require corresponding mutant host strains: e. g. D-alanine racemase (from Bacillus), URA3 (from S. cerevisiae or analogous genes from other yeasts), pyrG or pyrA (from A. nidulans or A. niger), argB (from A. nidulans or A. niger) or trpC. In a preferred embodiment the selection marker is deleted from the transformed host cell after introduction of the expression construct so as to obtain transformed host cells capable of producing the compound of interest or a compound involved in the synthesis of a compound of interest which are free of selection marker genes.

Expression of proteins in prokaryotes is often carried out in with vectors containing constitutive or inducible promoters directing the expression of either fusion or non-fusion proteins. Fusion vectors add a number of amino acids to a protein encoded therein, e.g. to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein.

The present invention also provides a host cell comprising a nucleic acid or an expression vector as disclosed herein. A suitable host cell may be a mammalian, insect, plant, fungal, or algal cell, or a bacterial cell.

The host cell may be a prokaryotic cell. Preferably, the prokaryotic host cell is bacterial cell. The term “bacterial cell” includes both Gram-negative and Gram-positive microorganisms. Suitable bacteria may be selected from e.g. Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces. Preferably, the bacterial cell is selected from the group consisting of B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Pseudomonas fluorescence, Paracoccus denitrificans, E. coli, C. glutamicum, Staphylococcus carnosus, Streptomyces lividans, Sinorhizobium melioti and Rhizobium radiobacter.

In one preferred embodiment of the invention the host cell deficient in the essential gene coding for the essential polypeptide is a prokaryotic cell, preferably a bacterial cell, more preferably a bacterial cell belonging to the genus Bacillus, Escherichia (such as Escherichia coli), Pseudomonas, Lactobacillus.

In a preferred embodiment of the invention the bacterial host cell may additionally contain modifications, e.g. the bacterial host cell may be deficient in genes which are detrimental to the production, recovery and/or application of the compound of interest, e.g. a compound of interest being a polypeptide, e.g. an enzyme. In a preferred aspect the bacterial host cell is a protease deficient host cell, more preferably it is a Bacillus host cell deficient in the gene aprE coding for extracellular alkaline protease and deficient in the gene nprE coding for extracellular neutral metalloprotease. In another preferred aspect the Bacillus host cell is further deficient in one or more proteases coded by the genes selected from the group consisting of: nprB, vpr, epr, wprA, mpr, bpr. In another preferred aspect the bacterial host cell does not produce spores and or is deficient in a sporulation related gene such as e.g. spoOA, spoIISA, sigE, sigF, spoIISB, spoIIE, sigG, spoIVCB, spoIIIC, spoIIGA, spoIIAA, spoIVFB, spoIIR, spoIIIJ. In yet another preferred aspect the Bacillus host cell is deficient in the gene amyE coding for α-amylase. In yet another preferred aspect the Bacillus host cell, more preferably a Bacillus subtilis host cell, is deficient in aprE, nprE, amyE and does not produce spores. In a more preferred embodiment the Bacillus host cell is BS154, CBS136327 or a derivative thereof.

According to an embodiment, the host cell according to the invention is a eukaryotic host cell. Preferably, the eukaryotic cell is a mammalian, insect, plant, fungal, or algal cell. Preferred mammalian cells include e.g. Chinese hamster ovary (CHO) cells, COS cells, 293 cells, PerC6 cells, and hybridomas. Preferred insect cells include e.g. Sf9 and Sf21 cells and derivatives thereof.

The eukaryotic cell may be a fungal cell, for example a yeast cell, such as a cell of the genus Candida, Hansenula, Kluyveromyces, Pichia, Saccharomyces, Schizosaccharomyces, or Yarrowia. More specifically, a yeast cell may be from Kluyveromyces lactis, Saccharomyces cerevisiae, Hansenula polymorpha, Yarrowia lipolytica and Pichia pastoris, Candida krusei.

Filamentous fungi include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma. Preferred filamentous fungal cells belong to a species of an Acremonium, Aspergillus, Chrysosporium, Myceliophthora, Penicillium, Talaromyces, Rasamsonia, Thielavia, Fusarium or Trichoderma genus, and most preferably a species of Aspergillus niger, Acremonium alabamense, Aspergillus awamori, Aspergillus foetidus, Aspergillus sojae, Aspergillus fumigatus, Talaromyces emersonii, Rasamsonia emersonii, Aspergillus oryzae, Chrysosporium lucknowense, Fusarium oxysporum, Myceliophthora thermophila, Trichoderma reesei, Thielavia terrestris or Penicillium chrysogenum. A more preferred filamentous fungal host cell belongs to the genus Aspergillus, more preferably the host cell belongs to the species Aspergillus niger. When the host cell according to the invention is an Aspergillus niger host cell, the host cell preferably is CBS 513.88, CBS124.903 or a derivative thereof.

Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), Agricultural Research Service Patent Culture Collection, Northern Regional Research Center (NRRL), and All-Russian Collection of Microorganisms of Russian Academy of Sciences, (abbreviation in Russian—VKM, abbreviation in English—RCM), Moscow, Russia. Useful strains in the context of the present invention may be Aspergillus niger CBS 513.88, CBS124.903, Aspergillus oryzae ATCC 20423, IFO 4177, ATCC 1011, CBS205.89, ATCC 9576, ATCC14488-14491, ATCC 11601, ATCC12892, P. chrysogenum CBS 455.95, P. chrysogenum Wisconsin54-1255(ATCC28089), Penicillium citrinum ATCC 38065, Penicillium chrysogenum P2, Thielavia terrestris NRRL8126, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Myceliophthora thermophila C1, Garg 27K, VKM-F 3500 D, Chrysosporium lucknowense C1, Garg 27K, VKM-F 3500 D, ATCC44006 and derivatives thereof.

A host cell may be a recombinant or transgenic host cell. The host cell may be genetically modified with a nucleic acid construct or expression vector as disclosed herein with standard techniques known in the art, such as electroporation, protoplast transformation or conjugation for instance as disclosed in Sambrook & Russell, Molecular Cloning: A Laboratory Manual, 3rd Ed., CSHL Press, Cold Spring Harbor, N.Y., 2001.

The invention also relates to a process for the production of a polypeptide of the invention comprising cultivating a host cell in a suitable fermentation medium under conditions conducive to the production of the polypeptide and producing the polypeptide. A skilled person in the art understands how to perform a process for the production of a polypeptide as disclosed herein depending on a host cell used, such as pH, temperature and composition of a fermentation medium. Host cells can be cultivated in shake flasks, or in fermenters having a volume of 0.5 or 1 litre or larger to 10 to 100 or more cubic metres. Cultivation may be performed aerobically or anaerobically depending on the requirements of a host cell.

Advantageously a polypeptide as disclosed herein is recovered or isolated from the fermentation medium.

The present invention further provides a composition comprising a polypeptide according to the invention. The composition may optionally comprise other ingredients such as, for example, a carrier, an excipient or a further enzyme, such as an auxiliary enzyme. A composition of the invention may a polypeptide of the invention and one or more further asparaginases. The one or more further asparaginases may be a second or further polypeptide of the invention.

A composition of the invention may comprise a polypeptide of the invention and at least one dough ingredient.

Dough ingredients include, without limitation, (cereal) flour, egg, water, salt, sugar, flavours, fat (including butter, margarine, oil and shortening), baker's yeast, a chemical leavening system such as a combination of an acid (generating compound) and bicarbonate, milk (including liquid milk and milk powder), soy flour, oxidants (including ascorbic acid, bromate and azodicarbonamide (ADA), reducing agents (including L-cysteine), emulsifiers (including mono/di glycerides, mono glycerides such as glycerol monostearate (GMS), sodium stearoyl lactylate (SSL), calcium stearoyl lactylate (CSL), polyglycerol esters of fatty acids (PGE) and diacetyl tartaric acid esters of mono- and diglycerides (DATEM) propylene glycol monostearate (PGMS), lecithin), gums (including guargum and xanthangum), flavours, acids (including citric acid, propionic acid), starch, modified starch, humectants (including glycerol) and preservatives

Cereals include maize, rice, wheat, barley, sorghum, millet, oats, rye, triticale, buckwheat, quinoa, spelt, einkorn, emmer, durum and kamut.

The preparation of a dough from dough ingredients is well known in the art and includes mixing of said ingredients and optionally one or more moulding and fermentation steps.

Preparing a dough according to the invention may comprise the step of combining a polypeptide of the invention or a composition of the invention and at least one dough ingredient.

Combining includes, without limitation, adding a polypeptide or a composition of the invention to the at least one component indicated herein, adding the at least one component indicated herein to a polypeptide or a composition of the invention, mixing a polypeptide or a composition of the invention and the at least one component indicated herein.

A Composition May Comprise a Polypeptide of the Invention and One or More Additional Enzymes

In such a composition of the invention, the additional enzyme may include including an alpha-amylase, such as a fungal alpha-amylase (which may be useful for providing sugars fermentable by yeast and retarding staling), beta-amylase, a cyclodextrin glucanotransferase, a protease, a peptidase, in particular, an exopeptidase (which may be useful in flavour enhancement), transglutaminase, triacyl glycerol lipase (which may be useful for the modification of lipids present in the dough or dough constituents so as to soften the dough), galactolipase, phospholipase, cellulase, hemicellulase, in particular a pentosanase such as xylanase (which may be useful for the partial hydrolysis of pentosans, more specifically arabinoxylan, which increases the extensibility of the dough), protease (which may be useful for gluten weakening in particular when using hard wheat flour), protein disulfide isomerase, e.g., a protein disulfide isomerase as disclosed in WO 95/00636, glycosyltransferase, peroxidase (which may be useful for improving the dough consistency), laccase, or oxidase, hexose oxidase, e.g., a glucose oxidase, aldose oxidase, pyranose oxidase, lipoxygenase or L-amino acid oxidase (which may be useful in improving dough consistency) or a protease.

In a composition of the invention, the additional enzyme may be a lipolytic enzyme.

In a composition of the invention, the additional enzyme may be an alpha amylase.

In a composition of the invention, the additional enzyme may be a copper-dependent lytic polysaccharide monooxygenase (GH61).

A lipolytic enzyme, also referred to herein as a lipase, is an enzyme that hydrolyses triacylglycerol and/or galactolipid and/or phospholipids. The specificity of the lipase can be shown through in vitro assay making use of appropriate substrate, for example triacylglycerol lipid, phosphatidylcholine and digalactosyldiglyceride, or preferably through analysis of the reactions products that are generated in the dough during mixing and fermentation.

The triacyl glycerol lipase may be a fungal lipase, preferably from Rhizopus, Aspergillus, Candida, Penicillum, Thermomyces, or Rhizomucor. In an embodiment the triacyl glycerol lipase is from Rhyzopus, in a further embodiment a triacyl glycerol lipase from Rhyzopus oryzae is used. Optionally a combination of two or more triacyl glycerol lipases may be used

In a composition of the invention, the additional enzyme may be a phospholipase.

In a composition of the invention, the additional enzyme may be a galactolipase.

In a composition of the invention, the additional enzyme may be an enzyme having both phospholipase and galactolipase activity.

Typically, a composition of the invention comprises a compound with which a polypeptide of the invention may be formulated. An excipient as used herein is an inactive substance formulated alongside with a polypeptide as disclosed herein, for instance sucrose or lactose, glycerol, sorbitol or sodium chloride. A composition comprising a polypeptide as disclosed herein may be a liquid composition or a solid composition. A liquid composition usually comprises water. When formulated as a liquid composition, the composition usually comprises components that lower the water activity, such as glycerol, sorbitol or sodium chloride (NaCl). A solid composition comprising a polypeptide as disclosed herein may comprise a granulate comprising the enzyme or the composition comprises an encapsulated polypeptide in liquid matrices like liposomes or gels like alginate or carrageenans. There are many techniques known in the art to encapsulate or granulate a polypeptide or enzyme (see for instance G. M. H. Meesters, “Encapsulation of Enzymes and Peptides”, Chapter 9, in N. J. Zuidam and V. A. Nedovi\grave{c} (eds.) “Encapsulation Technologies for Active Food Ingredients and food processing” 2010).

A composition of the invention may also comprise a carrier comprising a polypeptide of the invention. A polypeptide as disclosed herein may be bound or immobilized to a carrier by known technologies in the art.

A polypeptide or composition of the invention may be provided in a liquid form, to allow easy dispersion on the surface of the product, but dry powdered forms are also possible. Irrespective of the formulation of the polypeptide or composition, any additives and stabilizers known to be useful in the art to improve and/or maintain the enzyme's activity can be applied. When the polypeptide or composition is contained in a liquid form, it may be applied to a food product or an intermediate form of a food product by any conceivable method, for instance by soaking or spraying.

The present invention also relates to a process for preparing a composition comprising a polypeptide of the invention, which may comprise spray-drying a fermentation medium comprising the polypeptide, or granulating, or encapsulating a polypeptide of the invention, and preparing the composition.

A polypeptide according to the invention or a composition of the invention (comprising a said polypeptide) may be used in the production of a food product. That is to say, the invention provides use of a polypeptide of the invention, a polypeptide obtainable by a process of the invention for the preparation of a polypeptide or a composition of the invention in the production of a food product.

The term “food product” is defined to include both food stuffs for human consumption and food stuffs for animal consumption. Hence the term “food product” should be taken to mean “food, pet food or feed” throughout this document. An example of a food product is a baked product.

Thus, a polypeptide according to the invention or a composition of the invention may be used to reduce the amount of acrylamide formed in a thermally processed food product based on an asparagine-containing raw material. That is to say, the invention provides use of a polypeptide of the invention, a polypeptide obtainable by a process of the invention for the preparation of a polypeptide or a composition of the invention to reduce the amount of acrylamide formed in a thermally processed food product based on an asparagine-containing raw material.

A polypeptide or composition of the invention may, for example, be used in a process for the production of a food product involving at least one heating step, comprising adding one or more asparaginase enzymes to an intermediate form of said food product in said production process whereby the enzyme is added prior to or during said heating step in an amount that is effective in reducing the level of asparaginase that is present in said intermediate form of said food product.

That is to say, the invention provides a process for the production of a food product involving at least one heating step, which process comprises adding a polypeptide of the invention, a polypeptide obtainable by a process of the invention for the preparation of a polypeptide or a composition of the invention to an intermediate form of said food product in said production process, wherein the enzyme is added prior to or during said heating step in an amount that is effective in reducing the level of asparagine that is present in said intermediate form of said food product.

An amount that is effective in reducing the level of asparagine that is present in said intermediate form of said food product, also referred to as effective amount, includes an amount of asparaginase (in the form of a polypeptide or composition of the invention) of from about 0.1 to about 100 ASPU/g dry ingredient, more preferably from about 0.1 to 50 ASPU/g dry ingredient or from about 0.1 to 25 ASPU/g dry ingredient.

In an aspect, the asparginase is applied as an aqueous solution wherein the asparaginase is present in an amount of from about 0.1 to about 100 ASPU/g dry ingredient, such as from about 0.1 to about 50 ASPU/g dry ingredient, for example from about 0.1 to about 25 ASPU/g dry ingredient.

An amount that is effective in reducing the level of asparagine that is present in said intermediate form of said food product, also referred to as effective amount, may be determined in terms of the amount of asparaginase protein added (i.e. amount of protein/g ingredient. The amount to be added may depend on the specific activity of the asparaginase and may readily be determined by the person skilled in the art.

In the context of the present invention, 1 ASPU is defined as the amount of asparaginase that liberates one micromole of ammonia per minute from L-asparagine measured under the conditions of the assay as specified in the Examples. Asparaginase activity (in ASPU units) may be determined by measuring the rate of hydrolysis of L-asparagine to L-aspartic acid and ammonia. The liberated ammonia subsequently reacts with phenol nitroprusside and alkaline hypochlorite resulting in a blue color (Berthelot reaction). The activity of asparaginase may be determined by measuring absorbance of the reaction mixture at 630 nm.

The heating step in the process of the invention is one in which acrylamide may be formed should the intermediate form of the food product comprise asparagine. Such a heating step may be a frying or a baking step, for example.

Typically, the temperature of such a heating step is such that an asparaginase of the invention will be added to an intermediate form of the food product prior to the heating step. However, it may be possible to add the asparaginase to the intermediate form of the food during the heating step.

Food production processes may though have additional heating steps which take place prior to the heating step of a process of the invention in which acrylamide may be formed (in the event that asparagine is present). For example, in the production of French fries, blanching is a common unit operation which is typically in hot water (65-80° C.) for from 10 to 30 minutes. This is disadvantageous for treatment with asparaginase which is not thermophilic as the high temperature may inactivate the enzyme. Accordingly, an additional unit operation process at a lower temperature would need to be used. However, availability of a thermophilic asparaginase, which has a high temperature optimum and is thermostable may allow blanching to be combined with enzyme treatment. Accordingly, an asparaginase of the present invention may permit food production processes with fewer unit operations.

A process of the invention is disclosed in WO04/030468 which process and all its preferences are herein incorporated by reference. Also in WO04/026043 suitable processes are described wherein the asparaginase according to the invention could be used. The processes disclosed in WO04/026043 and all preferences disclosed are herein incorporated by reference.

An intermediate form of the food product is defined herein as any form that occurs during the production process prior to or during obtaining the final form of the food product. The intermediate form may comprise the individual raw materials used and/or mixture thereof and/or mixtures with additives and/or processing aids, or subsequently processed form thereof. For example, for the food product bread, the intermediate forms comprise for example wheat, wheat flour, the initial mixture thereof with other bread ingredients such as for example water, salt, yeast and bread improving compositions, the mixed dough, the kneaded dough, the leavened dough and the partially baked dough. For example for several potato-based products, dehydrated potato flakes or granules are intermediate products, and corn masa is an intermediate product for tortilla chips.

The food product may be made from at least one raw material that is of plant origin, for example potato, tobacco, coffee, cocoa, rice, cereal, for example wheat, rye corn, maize, barley, groats, buckwheat and oat. Wheat is here and hereafter intended to encompass all known species of the Triticum genus, for example aestivum, durum and/or spelta. Also food products made from more than one raw material or intermediate are included in the scope of this invention, for example food products comprising both wheat (flour and/or starch) and potato.

Examples of food products in which the process according the invention can be suitable for are any flour based products—for example bread, pastry, cake, pretzels, bagels, Dutch honey cake, cookies, gingerbread, gingercake and crispbread —, and any potato-based products—for example French fries, pommes frites, potato chips, croquettes.

The term food product includes without limitation a potato product, potato flakes, potato chips, potato crisps, French fries, hash browns, roast potatoes, breakfast cereals, infant cereals, crisp bread, muesli, biscuits, crackers, snack products, tortilla chips, corn chips, roasted nuts, rice crackers, Japanese “senbei”, wafers, waffles, hot cakes, pancakes, pretzels, salt or salty sticks, potato pellets and extruded potato snacks.

The term food product also includes without limitation cereal based food products, such as breakfast cereals and cereal snacks.

Potato flakes may be manufactured by drying cooked potato mash with drum drying. A polypeptide or composition of the invention may be be added at the mix and transfer stage. Process steps and times are typically as follows: potatoes are cooked (optionally in multiple stages) for from 15 minutes to 1 hour; and mixed and transferred to a drum dryer for from 5 to 30 minutes.

In typical industrial production of French fries, potatoes are initially washed, sorted, steam peeled and cut. Following cutting, the potato sticks may be blanched for from 5 to 60 minutes, optionally in 2 to 3 sequential steps. Blanching may be carried out to inactivate any endogenous enzymes in the potato, to partially cook the potato and/or to leach out reducing sugars to prevent excessive browning of the final product. Following blanching, the potato strips may quickly be dipped, e.g. for from 20 to 180 seconds, in a warm phosphate salt solution, e.g., a warm solution of sodium acid pyrophosphate (SAPP), to prevent greying of the final product. The dip may optionally be combined with a dip in glucose for reaching the desired colour. The potatoes may be dried in a drier with hot circulating air at from 45 to 95° C. for from 5 to 20 minutes giving a weight loss of form 5 to 25%. Finally, the potato sticks may be parfried before being quick-frozen and packed. Final frying is then carried out at a food service or by a consumer.

Breakfast cereals are a diverse range of products that may be processed in a number of ways. A polypeptide or composition of the invention may be used in the production of, for example, pressure cooked (often also referred to as batch processed) breakfast cereals, extruded breakfast cereals or shredded breakfast cereals.

Typically, the ingredients are mixed and then cooked within a pressure cooker. Depending on the scale of manufacture, the time taken to raise the temperature of the ingredients to above 80° C. is typically in the range of 10 to 60 minutes.

According to the invention, there is provided a dough which comprises a polypeptide of the invention, a polypeptide obtainable by a process of the invention for the preparation of a polypeptide or a composition of the invention.

The invention also provides a method for the preparation of such a dough. Such a method may comprise the step of:
combining: (i) a polypeptide of the invention, a polypeptide obtainable by a process of the invention for the preparation of a polypeptide or a composition of the invention; and (ii) at least one dough ingredient.

A method for the preparation of a baked product may thus comprise baking or frying a dough of the invention.

Dough is usually made using basic dough ingredients including (cereal) flour, such as wheat flour or rice flour, water and optionally salt. For leavened products, primarily baker's yeast is used next to chemical leavening systems such as a combination of an acid (generating compound) and bicarbonate.

The term dough herein includes a batter. A batter is a semi-liquid mixture, being thin enough to drop or pour from a spoon, of one or more flours combined with liquids such as water, milk or eggs used to prepare various foods, including cake and wafers.

The dough may be made using a mix including a cake mix, a biscuit mix, a brownie mix, a bread mix, a pancake mix and a crepe mix.

The term dough includes frozen dough, which may also be referred to as refrigerated dough. There are different types of frozen dough; that which is frozen before proofing and that which is frozen after a partial or complete proofing stage. The frozen dough is typically used for manufacturing baked products including without limitation biscuits, breads, bread sticks and croissants.

The term baked product includes, bread containing from 2 to 30 wt % sugar, fruit containing bread, breakfast cereals, cereal bars, eggless cake, soft rolls and gluten-free bread. Gluten free bread herein and herein after is bread than contains at most 20 ppm gluten. Several grains and starch sources are considered acceptable for a gluten-free diet. Frequently used sources are potatoes, rice and tapioca (derived from cassava). Baked product includes without limitation tin bread, loaves of bread, twists, buns, such as hamburger buns or steamed buns, chapati, rusk, dried steam bun slice, bread crumb, matzos, focaccia, melba toast, zwieback, croutons, soft pretzels, soft and hard bread, bread sticks, yeast leavened and chemically-leavened bread, laminated dough products such as Danish pastry, croissants or puff pastry products, muffins, Danish bagels, confectionery coatings, crackers, wafers, pizza crusts, tortillas, pasta products, crepes, waffles and par-baked products. An example of a par-baked product includes, without limitation, partially baked bread that is completed at point of sale or consumption with a short second baking process.

The bread may be white or brown pan bread and may for example be manufactured using a so called American style Sponge and Dough method or an American style Direct method.

The bread may be a floor bread, i.e. a bread which is baked on an oven plate.

The term tortilla herein includes corn tortilla and wheat tortilla. A corn tortilla is a type of thin, flat bread, usually unleavened made from finely ground maize (usually called “corn” in the United States). A flour tortilla is a type of thin, flat bread, usually unleavened, made from finely ground wheat flour. The term tortilla further includes a similar bread from South America called arepa, though arepas are typically much thicker than tortillas. The term tortilla further includes a laobing, a pizza-shaped thick “pancake” from China and an Indian Roti, which is made essentially from wheat flour. A tortilla usually has a round or oval shape and may vary in diameter from about 6 to over 30 cm.

The baked product may be a crusty bread having a crispy crust and a soft core. Examples of crusty bread include, but are not limited to, baguette, flute, pistolet, ciabatta, batard, Kaiser roll, hard roll, panini and maraguetta.

Raw materials as cited above are known to contain substantial amounts of asparagine which is involved in the formation of acrylamide during the heating step of the production process. Alternatively, the asparagine may originate from other sources than the raw materials e.g. from protein hydrolysates, such as yeast extracts, soy hydrolysate, casein hydrolysate and the like, which are used as an additive in the food production process. A preferred production process is the baking of bread and other baked products from wheat flour and/or flours from other cereal origin. Another preferred production process is the deep-frying of potato chips from potato slices.

Preferred heating steps are those at which at least a part of the intermediate food product, e.g. the surface of the food product, is exposed to temperatures at which the formation of acrylamide is promoted, e.g. 110° C. or higher, 120° C. or higher temperatures. The heating step in the process according to the invention may be carried out in ovens, for instance at a temperature between 180-220° C., such as for the baking of bread and other bakery products, or in oil such as the frying of potato chips, for example at 160-190° C.

Following application of the enzyme to a product, a certain processing time is required to allow the enzyme to act before the food is heated, because a substantial reduction of the amino acids capable of generating acrylamide must be obtained, and because the heating step will generally inactivate the enzyme. Generally the processing time will take at most 2 hours, preferably at most 1.5 hour and most preferably at most 1 hour. In general processing times of at least 2 minutes can be reached. Preferably, the processing time is between 2 minutes and 2 hours, more preferably between 5 minutes and 1.5 hours, and most preferably between 10 minutes and 1 hour.

It is to be understood that the more enzyme is added a shorter processing time can suffice for the enzyme to reach the desired effect and vice versa.

In another aspect, the invention provides food products obtainable by the process of the invention as described herein or by the use of a polypeptide of the invention to produce food products. These food products may be characterized by significantly reduced acrylamide levels in comparison with the food products obtainable by production processes that do not comprise adding a polypeptide of the invention in an amount that is effective in reducing the level of amino acids which are involved in the formation of acrylamide during a heating step. The process according to the invention may be used to obtain, for example, a decrease of the acrylamide content of the produced food product by preferably more than 50%, more preferably more than 20%, even more preferably 10% and most preferably more than 5% as compared to a food product obtained using the same process in which a polypeptide of the invention is not used.

An additional application for a polypeptide according to the invention is in the therapy of tumours. The metabolism of tumour cells requires L-asparagine, which can quickly be degraded by asparaginases. The asparaginase according to the invention can also be used as an adjunct in treatment of some human leukaemia. Administration of asparaginase in experimental animals and humans leads to regression of certain lymphomas and leukemia. Therefore, the invention provides a polypeptide of the invention, a polypeptide obtainable by a process of the invention for the preparation of a polypeptide or a composition of the invention for use in a method of treatment of the human or animal body by therapy, for example in the treatment of tumors, such as in the treatment of lymphomas or leukaemia in animals or humans.

Standard genetic techniques, such as overexpression of enzymes in the host cells, genetic modification of host cells, or hybridisation techniques, are known methods in the art, such as described in Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Methods for transformation, genetic modification etc of fungal host cells are known from e.g. EP-A-0 635 574, WO 98/46772, WO 99/60102 and WO 00/37671, WO90/14423, EP-A-0481008, EP-A-0635 574 and U.S. Pat. No. 6,265,186.

### EMBODIMENTS OF THE INVENTION

1. A polypeptide having asparaginase activity selected from the group consisting of:
   i. a polypeptide having an amino acid sequence comprising the mature polypeptide sequence of SEQ ID NO: 1;
   ii. a polypeptide comprising an amino acid sequence that has at least 50% sequence identity with the mature polypeptide sequence of SEQ ID NO: 1;
   iii. a polypeptide encoded by a nucleic acid comprising a sequence that hybridizes under medium stringency conditions to the complementary strand of the mature polypeptide encoding sequence of SEQ ID NO: 2; and
   iv. a polypeptide comprising an amino acid sequence encoded by a nucleic acid that has at least 50% sequence identity to the mature polypeptide coding sequence of SEQ ID NO: 2.
2. A polypeptide that is an isolated, substantially pure, pure, recombinant, synthetic or variant polypeptide of the polypeptide of embodiment 1.
3. A polypeptide according to embodiment 1 or 2 which is derivable from Thermoanaerobacter tengcongensis.
4. A composition comprising a polypeptide according to any one of embodiments 1 to 3.
5. A composition according to embodiment 4, comprising a carrier, an excipient, or an auxiliary enzyme and/or a dough ingredient.
6. A nucleic acid encoding an asparaginase which comprises a sequence that has at least 50% sequence identity to the mature polypeptide encoding sequence of SEQ ID NO: 2.
7. A nucleic acid that is an isolated, substantially pure, pure, recombinant, synthetic or variant nucleic acid of a nucleic acid of embodiment 6
8. An expression vector comprising a nucleic acid according to embodiment 6 or 7 operably linked to one or more control sequences that direct expression of the polypeptide in a host cell.
9. A recombinant host cell comprising a nucleic acid according to embodiment 6 or 7 or an expression vector according to embodiment 8.
10. A method for the preparation of a polypeptide according to embodiments 1 to 3, which method comprises:
   cultivating a host cell according to embodiment 9 in a suitable fermentation medium under conditions that allow for expression of the polypeptide; and, optionally, recovering the polypeptide.
11. Use of a polypeptide according to any one of embodiments 1 to 3, a polypeptide obtainable by a process according to embodiment 10 or a composition according to embodiment 4 or 5 in the production of a food product.
12. Use of a polypeptide according to any one of embodiments 1 to 3, a polypeptide obtainable by a process according to embodiment 10 or a composition according to embodiment 4 or 5 to reduce the amount of acrylamide formed in a thermally processed food product based on an asparagine-containing raw material.
13. A process for the production of a food product involving at least one heating step, which process comprises adding a polypeptide according to any one of embodiments 1 to 3, a polypeptide obtainable by a process according to embodiment 10 or a composition according to embodiment 4 or 5 to an intermediate form of said food product in said production process, wherein the enzyme is added prior to or during said heating step in an amount that is effective in reducing the level of asparagine that is present in said intermediate form of said food product.
14. A food product obtainable by the process according to embodiment 13 or by the use according to embodiment 11 or 12.
15. A dough comprising a polypeptide according to any one of embodiments 1 to 3, a polypeptide obtainable by a process according to embodiment 10 or a composition according to embodiment 4 or 5.
16. A method for the preparation of a dough, which method comprises combining: a polypeptide according to any one of embodiments 1 to 3, a polypeptide obtainable by a process according to embodiment 10 or a composition according to embodiment 4 or 5; and at least one dough ingredient.
17. A method for the preparation of a baked product, which method comprises the step of baking or frying a dough according to claim 15 or a dough obtainable by a method according to claim 16.
18. A polypeptide according to any one of embodiments 1 to 3, a polypeptide obtainable by a process according to embodiment 10 or a composition according to embodiment 4 or 5 for use in a method of treatment of the human or animal body by therapy.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

The present invention is further illustrated by the following Examples:

### EXAMPLES

### Materials and Methods

### General

Standard genetic techniques, such as overexpression of enzymes in the host cells, genetic modification of host cells, or hybridisation techniques, are known methods in the art, such as described in Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Methods for transformation, genetic modification etc of fungal host cells are known from e.g. EP-A-0 635 574, WO 98/46772, WO 99/60102 and WO 00/37671, WO90/14423, EP-A-0481008, EP-A-0635 574 and U.S. Pat. No. 6,265,186.

Water is Milli-Q water where nothing else is specified.

### Asparaginase Activity Assay

Asparaginases catalyze the hydrolysis of L-asparagine to aspartic acid and ammonia. To determine asparaginase activity, first an enzymatic reaction is performed by incubating the enzyme with L-asparagine. After stopping the enzymatic reaction, the released ammonia can be detected in a second non-enzymatic reaction where the ammonium formed combines with phenol to obtain the metachromatic dye indophenol, which can be accurately quantified by spectrophotometric analysis.

### Enzyme and Standard Incubation

In a 96 well PCR microtiter plate (MTP), 20 μL appropriately diluted asparaginase or ammonia standard was added to 100 μL L-asparagine solution and after sealing of the plate incubated in a PCR machine at 50, 60, 70, 80, 90 or 100° C. for 15 min. Reactions were stopped by transferring the MTP on ice. After centrifugation (5 min at 3000 rpm) to remove condensation, 20 μL of the reaction mixtures was transferred to a new MTP containing 180 μL water per well and mixed by pipetting.

Ammonium sulfate was used as standard in the range of 0 to 3.9 g/I. L-Asparagine solution: L-Asparagine (10 g/L: >99% pure asparaginase) was dissolved in assay buffer (100 mM of MOPS at pH 7 or a mixed buffer system consisting of 50 mM citrate, 50 mM KH₂PO₄ and 40 mM sodiumpyrophosphate at pH 5-10 (pH was adjusted with HCl or NaOH)).

The amount of sample used in Example 2 is such that the absorbance obtained after the indophenol method should not exceed that of the highest calibration point.

### Indophenol Method to Detect Ammonia

The formation of ammonia in enzyme reactions was quantified by adding 60 μL of the diluted reaction mixture to a new MTP containing per well 60 μL phenol nitroprusside solution (Sigma-Aldrich, product number: P6994). After addition of 60 μL alkaline hypochlorite (Sigma-Aldrich, product number: A1727) the plates were sealed and incubated for 15 min at 37° C. with shaking (750 rpm) on an Eppendorf thermomixer equipped with an MTP adapter. The absorbance was measured spectrophotometrically using a wavelength of 630 nm. The absorbance of the standards was plotted against the ammonia concentration in the standards, and the standard curve obtained was used to calculate the ammonia produced in the enzyme samples. The activity is given as micromole ammonia released per minute per ml sample (U/mL).

### UPLC-MSMS Method to Measure Asparagine

Potato dough samples (5 g dough in 100 ml 0.1 M HCl) and cereal (wheat) dough samples (10 g dough in 50 ml 0.1 M HCl) are centrifuged during 10 min at 14000 rpm and 100 μl of the supernatant is diluted with 100 μl of the internal labelled standard working solution (2 μg of L-asparagine-15N₂) into an Eppendorf reaction vial and mixed. For the calibration curve, 100 μl internal labelled standard working solution is added to a set of calibration solutions (100 μl; each solution containing 0.7-7 μg of Asn) in an Eppendorf reaction vial and mixed. For AccQtag derivatization, 10 μl of the abovementioned solutions (both sample and calibration solutions) are taken to which 70 μl of AccQ⋅Tag ultra reagent 1 (borate buffer) is added and mixed. To this mixture, 20 μl of AccQ⋅Tag ultra reagent 2 is added and mixed immediately. This solution is then transferred to an UPLC injection vial. UPLC-MS/MS analysis is performed according to Ref.1: ¹ Carolina Salazar, Jenny M. Armenta, Diego F. Codes and Vladimir Shulaev, Combination of an AccQ⋅Tag-Ultra Performance Liquid Chromatographic Method with Tandem Mass Spectrometry for the Analysis of Amino Acids,

### Methods In Molecular Biology (Clifton, N. J.), 828, 13-28 (2012)

### Example 1: Expression of a Putative New Asparaginase from Thermoanaerobacter tengcongensis in E. coli

In order to identify an L-asparaginase that function at high temperature, we tested >35 asparaginase candidates from thermophiles and hyper thermophiles (both bacteria and archaea). We first performed a blast search (using two different L-asparaginase sequence as query) to retrieve all candidate asparaginase genes from the NCBI database. Our search retrieved more than 1000 candidates as “hits”, suggesting that the search is comprehensive in covering all available L-asparaginase sequences.

Since it is not obvious by sequence alone which candidates would be functional at the desired high temperature, we further refined the hits using criteria:

Step 1.

We rationally selected for sequences originating from thermophiles and hyper thermophiles (primarily bacteria and archaea), after which 130 hits remained.

Step 2.

We then specifically selected for L-asparaginase that is not in the class of glutaminase-asparaginase (which usually contains an additional protein domain and thus is larger in size), by selecting for hits with sequence length between 300 to 400 amino acids. Protein alignment was then performed to confirm the identity of putative full length protein.

A synthetic gene based on the protein sequence of a putative asparaginase from Thermoanaerobacter tengcongensis (SEQ ID NO:1; accession number: WP_011025341.1) was designed by optimizing the gene codon usage for E. coli according to the algorithm of DNA2.0 (GeneGPS® technology).

For cloning purposes a DNA sequence containing a NdeI site CAT (was introduced at the 5′-end and a DNA sequence containing a stop codon and a AscI site TAACCTGCAGGGGCGCGCC was introduced at the 3′ end.

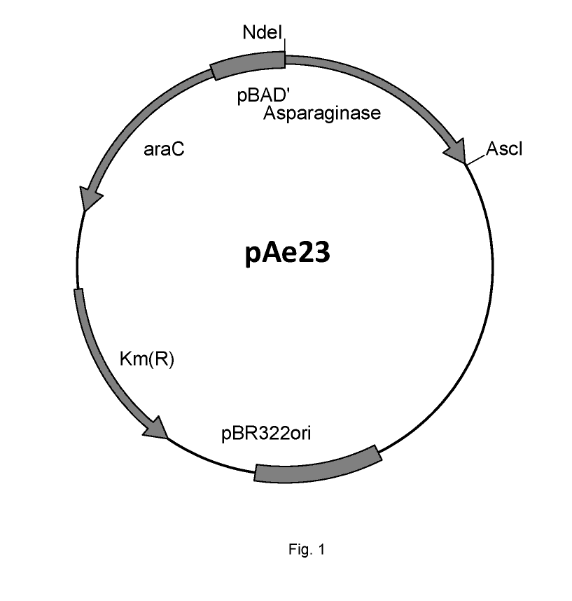

The synthetic DNA encoding the putative asparaginase (SEQ ID NO:2) was cloned via the 5′NdeI and 3′AscI restriction sites into an arabinose inducible E. coli expression vector, containing the arabinose inducible promoter P_{BAD} and regulator araC (Guzman J. Bac. 177:4121-4130, 1995), a kanamycin resistance gene Km(R) and the origin of replication ori327 from pBR322 (Watson, Gene. 70:399-403, 1988). Expression of the cloned gene may thus be induced by arabinose. The clones were sequence verified. The sequence of the final plasmid pAe7 is shown in FIG. 1.

The E. coli host RV308 (laclq-, su-, ΔlacX74, gal IS II::OP308, strA, http://www.ebi.ac.uk/ena/data/view/ERP005879) with additional deletions in ampC and araB was transformed using chemical competent cells (Z-Competent cells, prepared with the Mix and Go!E. coli transformation kit, Zymo Research, Irvine Calif., USA).

Correct transformants were pre cultured in 2xPY+neomycine (0/N). The preculture ( 1/100 vol) was used to inoculate the fermentation in MagicMedia™ E. coli expression medium (Thermo Fisher Scientific Inc), +neomycine (24 wells MTP, 3 ml volume, breathable seal, 550 RPM 80% RH), after 4 h growth at 30° C. 0.02% arabinose (final concentration) was used for induction of the asparaginase gene and incubation was continued at 20° C. for 48 h. Cell-pellets were frozen. Cell free extract (CFE) was prepared, incubation for 1 hour at 37° C. in lysis buffer ((Tris-HCl 50 mM, DNasel 0.1 mg/ml, lysozyme 2 mg/ml, MgSO4 25 μM). Cell debris was centrifuged and the CFE was transferred to a clean 96 well MTP covered by a silicone mat and stored at −20° C. until further characterization.

The putative asparaginase gene sequence originates from Caldanaerobacter subterraneus subsp. tengcongensis MB4 (=JCM 11007=NBRC100824=DSM15242) which is commercially available e.g. from the Japan Collection of Microorganisms. The type strain was described by Xue et al., 2001 (Thermoanaerobacter tengcongensis sp. nov., a novel anaerobic, saccharolytic, thermophilic bacterium isolated from a hot spring in Tengcong, China, Int J Syst Evol Microbiol. 2001 July; 51(Pt 4):1335-1341).

### Example 2: Biochemical Characterization of the Putative Asparaginase

2.1 Temperature Dependent Activity of Thermoanaerobacter tengcongensis Asparaginase

The temperature dependent activity of the Thermoanaerobacter tengcongensis putative asparaginase was determined by performing catalytic activity measurements at selected temperatures for 15 min at pH 7.0. Samples (CFE from Example 1) were diluted appropriately in 100 mM MOPS buffer pH 7.0. A 96 well PCR plate containing 100 μL L-asparagine solution (10 g/L) in 100 mM MOPS buffer pH 7.0 per well, was placed on ice and 20 μL of diluted sample was added. After sealing, the plate was incubated in a PCR cycler for 15 min at 50, 60, 70, 80, 90 or 100° C. Following incubation, the plate was immediately placed on ice to stop the enzymatic reaction. After cooling down, the plate was centrifuged (5 min at 3000 rpm) to remove condensation from the seal.

Next, the concentration of ammonia in the reactions was determined as follows. The reaction mixtures were diluted 10-fold by adding 20 μL to a new MTP containing 180 μL water per well and mixing by pipetting. 60 μL of the diluted mixture was then added to a new MTP containing 60 μL phenol nitroprusside solution per well. Finally, 60 μL alkaline hypochlorite was added to each well and the plate was sealed and incubated for 15 min at 37° C. After cooling to ambient temperature, the MTP plate was centrifuged (5 min at 3000 rpm) and the absorbance measured at 630 nm.

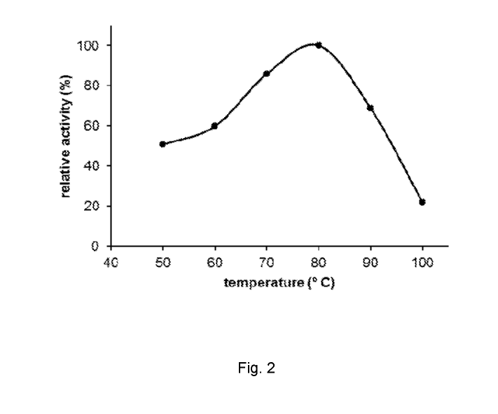

The relative activity is shown in FIG. 2. The Thermoanaerobacter tengcongensis asparaginase shows maximal catalytic activity at 80° C.

2.2 pH Dependent Activity of Thermoanaerobacter tengcongensis Asparaginase

The pH dependent activity of Thermoanaerobacter tengcongensis asparaginase was determined by performing catalytic activity measurements at selected pH values for 15 min at 70° C. Samples (CFE from Example 1) were diluted in water. A 96 well PCR plate containing 100 μL L-asparagine solution (10 g/L) in buffer (50 mM citrate, 50 mM Na2HPO4 and 40 mM sodium pyrophosphate, adjusted to pH 5, 6, 7, 8, 9 and 10) per well, was placed on ice and 20 μL of diluted sample was added. After sealing, the plate was incubated in a PCR cycler for 15 min at 70° C. Following incubation, the plate was immediately placed on ice to stop the enzymatic reaction. After cooling down, the plate was centrifuged (5 min at 3000 rpm) to remove condensation from the seal.

Next, the concentration of ammonia in the reactions was determined as follows. The reaction mixtures were diluted 10-fold by adding 20 μL to a new MTP containing 180 μL water per well and mixing by pipetting. 60 μL of the diluted mixture was then added to a new MTP containing 60 μL phenol nitroprusside solution per well. Finally, 60 μL alkaline hypochlorite was added to each well and the plate was sealed and incubated for 15 min at 37° C. with shaking (750 rpm). After cooling to ambient temperature, the MTP plate was centrifuged (5 min at 3000 rpm) and the absorbance measured at 630 nm.

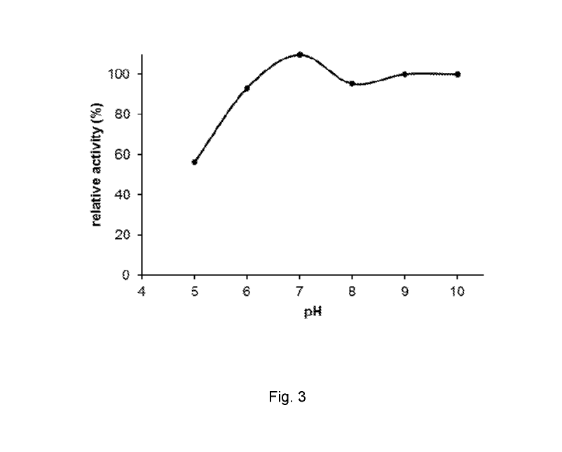

The relative activity is shown in FIG. 3. The Thermoanaerobacter tengcongensis asparaginase shows maximal catalytic activity at pH 7.

### Example 3: Use of the Asparaginase to Reduce Asparaginase in a Potato Mash as a Model for Potato Flake Manufacture

Potato flakes are made by drying hot potato mash. In this Example, potato flakes are rehydrated and heated to bring them back to the earlier stage of their manufacturing.

The asparaginase expressed from E. coli as described in Example 1 is used to treat the potato mash suitable for the production of potato flakes.

100 g of water is added to 30 g potato flakes. This mixture is heated for 45 minutes until a consistent temperature of 90° C. is reached. Enzyme is then added by mixing for 1 minute and the resulting mixture then held for 15 minutes at 90° C. The enzyme is inactivated by exactly weighing in approximately 5 g of potato mash into 100 ml of 0.1 M HCl.

Different dosage levels of enzyme, ranging from 1000 Units to 15000 ASPU/kg dry potato are tested. Asparagine levels are determined using UPLC-MSMS analysis and compared to the same food product made in the absence of enzyme.

The Thermoanaerobacter tengcongensis asparaginase performed well in this test.

### Example 4: Use of the Asparaginase in a Cereal Mix Suitable for Cereal Product Manufacturing

The asparaginase expressed from E. coli as described in Example 1 is used in the production of a food-product simulating the production of a breakfast cereal using two different protocols.

15 g of water is added to 35 g wholemeal flour and enzyme then added, followed by mixing for 4 minutes with a Siemens hand-blender. This mixture is then held for 35 minutes in a closed water bath at 90° C. The enzyme was inactivated by exactly weighing in approximately 10 g of cereal mix into 50 ml of 0.1 M HCl.

Different dosage levels of enzyme, ranging from 150 Units to 1500 ASPU/kg wholemeal flour are tested.

Asparagine levels are determined using UPLC-MSMS analysis and compared to the same food product made in the absence of enzyme.

The Thermoanaerobacter tengcongensis asparaginase performed well in this test.

### Example 5: Use of the Asparaginase to Reduce Asparaginase in a Potato Mash as a Model for Potato Flake Manufacture

Potato flakes are made by drying hot potato mash. In this Example, potato flakes are rehydrated and heated to bring them back to the earlier stage of their manufacturing.

The asparaginase expressed from E. coli as described in Example 1 is used to treat the potato mash suitable for the production of potato flakes. PreventASe XR™ is a non heat stable asparaginase enzyme used as control.

100 g of water was added to 30 g potato flakes and mixed for 1 minute to reach a temperature of 54 C+/−3° C. The mix was further heated from 55° C. to 90° C. for 14 minutes and then held for a further 15 minutes from 90° C. to a final mix temperature of 94-95° C.

The enzyme was inactivated by weighing in of 5 g potato mash into 100 ml of 0.1M HCl and shaken vigorously for two minutes.

Different dosage levels of enzyme, ranging from 1000 Units to 10000 Units per kg dry potato were tested. Asparagine levels were determined using UPLC-MSMS analysis and compared to the same food product made in the absence of enzyme. The results are shown in table 2 below.

TABLE 2

| | Amount of | | | |
|---|---|---|---|---|
| | asparagine | | | |
| | Enzyme | | Amount of | |
| | according to | % | asparagine | % |
| | example 1 (ASN | asparagine | PreventASe XR ™ | asparagine |
| Dosage | g/kg) | reduction | (ASN g/kg) | reduction |
| 0 | 1.173 | 0 | 1.199 | 0 |
| 1000 | 1.001 | 15% | 1.144 | 5% |
| 1250 | 0.429 | 63% | 1.177 | 2% |
| 5000 | 0.165 | 86% | 1.144 | 5% |
| 7500 | 0.099 | 92% | 1.122 | 6% |
| 1000 | 0.088 | 93% | 1.177 | 2% |

### Example 6: Use of the Asparaginase in a Cereal Mix Suitable for Cereal Product Manufacturing

The asparaginase expressed from E coli in Example 1 was used in the production of a food-product simulating the production of a breakfast cereal.

37.5 g of water is added to 30 g wholemeal flour is mixed for 5 seconds, enzyme then added, and mixed for a further 55 seconds This mixture is then heated from approximately from 51° C. to 90° C. over 12 minutes. It was then held for a further 15 minutes to heat the mix from (or where the temperature rises from?) 90° C. to a final mix temperature of 94-95° C.

The enzyme was inactivated by weighing 10 g of cereal mix into 50 ml of 0.1M HCl and shaken vigorously for two minutes.

Different dosage levels of enzyme, ranging from 150 Units to 1500 ASPU/kg wholemeal flour were tested.

Asparagine levels were determined using UPLC-MSMS analysis and compared to the same food product made in the absence of enzyme. The results are shown in table 3 below.

TABLE 3

| | Amount of | | | |
|---|---|---|---|---|
| | asparagine | | | |
| | Enzyme | | Amount of | |
| | according to | % | asparagine | % |
| | example 1 (ASN | asparagine | PreventASe XR ™ | asparagine |
| Dosage | g/kg) | reduction | (ASN g/kg) | reduction |
| 0 | 0.2596 | 0 | 0.2508 | 0 |
| 150 | 0.22 | 16% | 0.2563 | 2% |
| 300 | 0.22 | 16% | 0.2453 | 3% |
| 600 | 0.1793 | 31% | 0.2422 | 3% |
| 900 | 0.1452 | 44% | 0.1518 | 40% |
| 1500 | 0.0418 | 84% | 0.2321 | 8% |

### Example 7: Use of the Asparaginase to Reduce Asparaginase in a Potato Mash as a Model for Potato Flake Manufacture

Potato flakes were made as in example 3, wherein the potato flakes were placed into a closed water bath having a water temperature of 94° C. and the hydrated potato mix held in water bath for 30 mins until the dough is 90° C.+/−3° C. Asparagine levels were determined using UPLC-MSMS analysis and compared to the same food product made in the absence of enzyme. The results are shown in table 4 below.

TABLE 4

| | Amount of | |
|---|---|---|
| | asparagine | |
| | Enzyme | |
| | according to | |
| | example 1 (ASN | % asparagine |
| Dosage | g/kg) | reduction |
| 0 | 2.8602 | 0 |
| 1000 | 2.2241 | 22% |
| 1250 | 1.3916 | 51% |
| 5000 | 0.5477 | 81% |
| 7500 | 1.3000 | 55% |
| 1000 | 0.3185 | 89% |

1. A polypeptide having asparaginase activity selected from the group consisting of:
i. a polypeptide having an amino acid sequence comprising the mature polypeptide sequence of SEQ ID NO: 1;
ii. a polypeptide comprising an amino acid sequence that has at least 50% sequence identity with the mature polypeptide sequence of SEQ ID NO: 1;
iii. a polypeptide encoded by a nucleic acid comprising a sequence that hybridizes under medium stringency conditions to the complementary strand of the mature polypeptide encoding sequence of SEQ ID NO: 2; and
iv. a polypeptide comprising an amino acid sequence encoded by a nucleic acid that has at least 50% sequence identity to the mature polypeptide coding sequence of SEQ ID NO: 2.

2. A polypeptide according to claim 1 which is derivable from Thermoanaerobacter tengcongensis.

3. A composition comprising: a polypeptide according to claim 1; and a carrier, an excipient, an auxiliary enzyme and/or a dough ingredient.

4. A nucleic acid encoding an asparaginase which comprises a sequence that has at least 50% sequence identity to the mature polypeptide encoding sequence of SEQ ID NO: 2.

5. An expression vector comprising a nucleic acid according to claim 4 operably linked to one or more control sequences that direct expression of the polypeptide in a host cell.

6. A recombinant host cell comprising a nucleic acid according to claim 4 or an expression vector comprising said nucleic acid operably linked to one or more control sequences that direct expression of the polypeptide in a host cell.

7. A method for the preparation of a polypeptide, which method comprises:
cultivating a host cell according to claim 6 in a suitable fermentation medium under conditions that allow for production of the polypeptide; and, optionally,
recovering the polypeptide.

8. A product comprising a polypeptide according to claim 1 or a composition thereof used in the production of a food product.

9. A product comprising a polypeptide according to claim 1 a composition thereof adapted to reduce the amount of acrylamide formed in a thermally processed food product based on an asparagine-containing raw material.

10. A production process for a food product involving at least one heating step, which process comprises adding a polypeptide according to claim 1 or a composition thereof to an intermediate form of said food product in said production process, wherein the enzyme is added prior to or during said heating step in an amount that is effective in reducing the level of asparagine that is present in said intermediate form of said food product.

11. A food product obtainable by the process according to claim 10.

12. A dough comprising a polypeptide according to claim 1 or a composition thereof.

13. A method for the preparation of a dough, which method comprises combining: a polypeptide according to claim 1 or a composition thereof; and at least one dough ingredient.

14. A method for the preparation of a baked product, which method comprises baking or frying a dough according to claim 12 or a dough obtainable by a process comprising combining: a polypeptide according to claim for a composition thereof and at least one dough ingredient.

15. A polypeptide according to claim 1 or a composition thereof for use in a method of treatment of the human or animal body by therapy.
